(19) United States
(12) Patent Application Publication
(10) Pub. No.: US 2014/0112447 A1
(43) Pub. Date: Apr. 24, 2014

(54) RADIATION IMAGING SYSTEM, INFORMATION PROCESSING METHOD IN RADIATION IMAGING SYSTEM, CONTROL APPARATUS, AND STORAGE MEDIUM

(71) Applicant: Canon Kabushiki Kaisha, Tokyo (JP)

(72) Inventor: Daiya Semba, Inagi-shi (JP)

(73) Assignee: Canon Kabushiki Kaisha, Tokyo (JP)

(21) Appl. No.: 14/051,678

(22) Filed: Oct. 11, 2013

(30) Foreign Application Priority Data
Oct. 19, 2012 (JP) 2012-232335

(51) Int. Cl. A61B 6/00

(52) U.S. Cl. CPC A61B 6/52
USPC 378/98; 378/91

(57) ABSTRACT

A radiation imaging system for imaging an object to obtain a radiation image based on an imaging protocol, the system comprising: a storage unit configured to store a radiation image, with image information indicating an attribute of the radiation image being added thereto; a copying unit configured to, upon receiving an instruction to copy a radiation image stored in the storage unit, copy the radiation image and image information added to the radiation image; and a changing unit configured to change an item, of items included in copied image information copied by the copying unit, which is unique to each of radiation images and different from each other, in accordance with a copied image, thereby changing the copied image information.

### BACKGROUND OF THE INVENTION

1. Field of the Invention

The present invention relates to a radiation imaging system, an information processing method in the radiation imaging system, a control apparatus, and a storage medium and, more particularly, to an information processing technique for copying a captured image in a radiation imaging system.

2. Description of the Related Art

Recently, hospital information systems have been constructed based on network connection in hospitals. When, for example, the operator determines that X-ray imaging is necessary, he/she inputs an examination instruction with an HIS (Hospital Information System) terminal. This instruction is transmitted to an RIS (Radiology Information System) in a radiology department as a request destination. This request information is called “examination information”. This examination information includes the name of the department to which the request source belongs, an examination item, and object information.

Upon receiving the examination information, the radiology department adds imaging conditions and the like to the information and outputs the resultant information to an X-ray imaging apparatus. The X-ray imaging apparatus executes X-ray imaging in accordance with the received examination information. The apparatus performs image processing determined in advance for each region for an image captured by the apparatus (captured image) and displays it on a display unit. The apparatus also adds the information to the examination information and outputs the resultant information to a PACS (Picture Archiving and Communication Systems) or outputs the information to a printer.

In this case, depending on received examination information, it is sometimes necessary to perform a plurality of different types of image processing for one captured image. If, for example, an examination item is “pneumoconiosis examination”, the apparatus applies image processing determined for pneumoconiosis examination independently of a captured image to which general image processing has been applied. In such a case, in general, the operator often applies the image processing determined for pneumoconiosis examination without processing the original captured image.

That is, if it is necessary to perform a plurality of different types of image processing for one captured image, the system copies the captured image and “image information” (information indicating the attributes of the captured image) added to the captured image. The system then performs different types of image processing for the copied image without processing the captured image as the copy source (see, for example, Japanese Patent Laid-Open No. 2009-70201).

However, when copying a captured image in the radiation imaging system, directly copying the captured image and its additional image information will sometimes pose a problem. This will be described below with reference to concrete examples.

For example, there are various types of relationships between captured images and “imaging protocols” which define imaging conditions and the contents of image processing for the generation of the captured images. When copying a captured image, it is necessary to consider these relationships.

More specifically, the relationships between captured images and imaging protocols include a case in which an imaging protocol is one-to-one linked to a captured image and a case in which an imaging protocol is linked to a plurality of captured images. In addition, consider one “stitch composite image” generated from a plurality of captured images (partial images). In this case, the imaging protocols used for the capturing of the respective partial images (that is, the plurality of imaging protocols) are linked to the stitch composite image. For this reason, when copying an image such as a captured image or stitch composite image, it is indispensable to perform processing corresponding to the correspondence relationship between the imaging protocol and the image.

In addition, the image information added to a captured image includes a mixture of an item which can be directly copied and an item which cannot be directly copied. When the image information is copied, it is necessary to perform further changing processing.

Consider, for example, output state information (information indicating whether the captured image has been output to the PACS or the like) of image information. If the copy source image has already been output, this information is set to “output state”. It is, however, necessary to change the image information of a copy image from “output state” to “non-output state”. If a unique instance number is assigned to each captured image as image information, it is necessary to assign a unique instance number different from that of the copy source image to a copy image.

Moreover, when copying an image and displaying a copy image, the above radiation imaging system needs to consider the following points.

When, for example, displaying a copy source image and a copy image in the form of a list, the system preferably allows the operator to discriminate each image as the copy source image or the copy image.

In addition, when the operator copies an image to perform different types of image processing for the copy source image and the copy image, he/she regards the system as operating in the operation form of immediately performing image processing for the copy image. For this reason, when image copying is complete, it is preferable that the copy image has been automatically selected as a target for which image processing is to be performed.

When calculating and displaying a radiation dose based on the image information added to a captured image, it is necessary to perform this processing, excluding the image information added to the copy image.

Furthermore, the radiation imaging system sometimes performs re-imaging operation upon generating a captured image. For this reason, an image to be copied includes a re-captured image in addition to a captured image. It is therefore necessary to give consideration to problems posed when copying the re-captured image.

For example, consider a case in which in a radiation imaging system designed to make an imaging protocol and a re-imaging protocol synchronously operate, re-imaging operation sets a captured image before re-imaging in an imaging failure state, and a new re-imaging protocol is added. In this case, when copying the captured image (or re-captured image), the system should perform processing so as to inhibit the imaging protocol (or re-imaging protocol) linked to the copy image from operating in synchronism with the imaging protocol (or the re-imaging protocol) linked to the copy source image.

As described above, when copying an image in the radiation imaging system, it is necessary to improve user's operability by individually coping with the problems unique to the radiation imaging system.

### SUMMARY OF THE INVENTION

The present invention has been made in consideration of the above problems, and provides a technique of improving user's operability when copying an image in a radiation imaging system.

According to one aspect of the present invention, there is provided a radiation imaging system for imaging an object to obtain a radiation image based on an imaging protocol, the system comprising: a storage unit configured to store a radiation image, with image information indicating an attribute of the radiation image being added thereto; a copying unit configured to, upon receiving an instruction to copy a radiation image stored in the storage unit, copy the radiation image and image information added to the radiation image; and a changing unit configured to change an item, of items included in copied image information copied by the copying unit, which is unique to each of radiation images and different from each other, in accordance with a copied image, thereby changing the copied image information, wherein the copying unit switches methods of copying an imaging protocol used for obtaining a radiation image for which the copy instruction has been issued, in accordance with a relationship between the radiation image and the imaging protocol.

Further features of the present invention will be apparent from the following description of exemplary embodiments with reference to the attached drawings.

### BRIEF DESCRIPTION OF THE DRAWINGS

FIG. 1 is a diagram showing the overall arrangement of an X-ray imaging system 101 according to an embodiment of the present invention;

FIG. 2 is a block diagram showing an example of the functional arrangement of an X-ray imaging control unit 112 in the X-ray imaging system 101 according to the first to third embodiments;

FIG. 3 is a view showing an example of the display window before image copying which is displayed on a display unit 113 in the X-ray imaging system 101 according to the first embodiment;

FIG. 4 is a view showing an example of the display window after image copying which is displayed on the display unit 113 in the X-ray imaging system 101 according to the first embodiment;

FIG. 5 is a flowchart showing a procedure for examination processing in an X-ray imaging apparatus 107 of the X-ray imaging system 101 according to the first to third embodiments;

FIG. 6 is a flowchart showing a procedure for image copying processing in the X-ray imaging apparatus 107 of the X-ray imaging system 101 according to the first to third embodiments;

FIG. 7 is a flowchart showing a procedure for image information changing processing in the X-ray imaging apparatus 107 of the X-ray imaging system 101 according to the first to third embodiments;

FIG. 8 is a view showing an example of the display window before image copying which is displayed on a display unit 113 in the X-ray imaging system 101 according to the second embodiment;

FIG. 9 is a view showing an example of the display window after image copying which is displayed on the display unit 113 in the X-ray imaging system 101 according to the second embodiment;

FIG. 10 is a view showing an example of the display window before image copying which is displayed on a display unit 113 in the X-ray imaging system 101 according to the third embodiment;

FIG. 11 is a view showing an example of the display window after image copying which is displayed on the display unit 113 in the X-ray imaging system 101 according to the third embodiment;

FIG. 12 is a block diagram showing an example of the functional arrangement of an X-ray imaging control unit 112 in an X-ray imaging system 101 according to the fourth embodiment;

FIG. 13 is a view showing an example of the display window before re-imaging preparation which is displayed on a display unit 113 in the X-ray imaging system 101 according to the fourth embodiment;

FIG. 14 is a view showing an example of the display window after re-imaging preparation which is displayed on the display unit 113 in the X-ray imaging system 101 according to the fourth embodiment;

FIG. 15 is a view showing an example of the display window after re-imaging which is displayed on the display unit 113 in the X-ray imaging system 101 according to the fourth embodiment;

FIG. 16 is a view showing an example of the display window displayed on the display unit 113 when an imaging failure state is switched after re-imaging in the X-ray imaging system 101 according to the fourth embodiment;

FIG. 17 is a view showing an example of the display window displayed on the display unit 113 after copying of a re-captured image in the X-ray imaging system 101 according to the fourth embodiment;

FIG. 18 is a view showing an example of the display window displayed on the display unit 113 upon switching of the imaging failure state of a copy source image or captured image in a re-imaging relationship with the copy source image after copying of a re-captured image in the X-ray imaging system 101 according to the fourth embodiment;

FIG. 19 is a view showing an example of the display window displayed on the display unit 113 upon switching of a copy image of a re-captured image to the imaging failure state in the X-ray imaging system 101 according to the fourth embodiment;

FIG. 20 is a view showing an example of the display window displayed on the display unit 113 after copying of an image before re-imaging in the X-ray imaging system 101 according to the fourth embodiment;

FIG. 21 is a view showing an example of the display window displayed on the display unit 113 upon switching of the imaging failure state of a copy source image or re-captured image in a re-imaging relationship with the copy source image after copying of a captured image before re-imaging in the X-ray imaging system 101 according to the fourth embodiment;

FIG. 22 is a view showing an example of the display window displayed on the display unit 113 when the imaging failure state of a copy image of an image before re-imaging is released in the X-ray imaging system 101 according to the fourth embodiment;

FIG. 23 is a flowchart showing a procedure for examination processing in an X-ray imaging apparatus 107 of the X-ray imaging system 101 according to the fourth embodiment;

FIG. 24 is a flowchart showing a procedure for re-imaging preparation processing in the X-ray imaging apparatus 107 of the X-ray imaging system 101 according to the fourth embodiment;

FIG. 25 is a flowchart showing a procedure for the processing of releasing the imaging failure state of a captured image in the X-ray imaging apparatus 107 of the X-ray imaging system 101 according to the fourth embodiment;

FIG. 26 is a flowchart showing a procedure for the processing to be performed when copying no image after re-imaging in the X-ray imaging apparatus 107 of the X-ray imaging system 101 according to the fourth embodiment;

FIG. 27 is a flowchart showing a procedure for the processing of switching a re-captured image to the imaging failure state in the X-ray imaging apparatus 107 of the X-ray imaging system 101 according to the fourth embodiment;

FIG. 28 is a flowchart showing a procedure for the processing to be performed when copying a re-captured image after re-imaging in the X-ray imaging apparatus 107 of the X-ray imaging system 101 according to the fourth embodiment;

FIG. 29 is a flowchart showing a procedure for image information changing processing in the X-ray imaging apparatus 107 of the X-ray imaging system 101 according to the fourth embodiment; and

FIG. 30 is a flowchart showing a procedure for the processing to be performed when a captured image before re-imaging is copied after re-imaging in the X-ray imaging apparatus 107 of the X-ray imaging system 101 according to the fourth embodiment.

### DESCRIPTION OF THE EMBODIMENTS

An exemplary embodiment(s) of the present invention will now be described in detail with reference to the drawings. It should be noted that the relative arrangement of the components, the numerical expressions and numerical values set forth in these embodiments do not limit the scope of the present invention unless it is specifically stated otherwise.

Each embodiment of the present invention will be described in detail below with reference to the accompanying drawings. Each embodiment to be described below will exemplify an imaging system using X-rays as radiation. However, the radiation to be used in the imaging system according to the present invention is not limited to X-rays, but may be electromagnetic waves, α-rays, β-rays, or γ-rays.

### First Embodiment

### 1. Arrangement of X-ray Imaging System

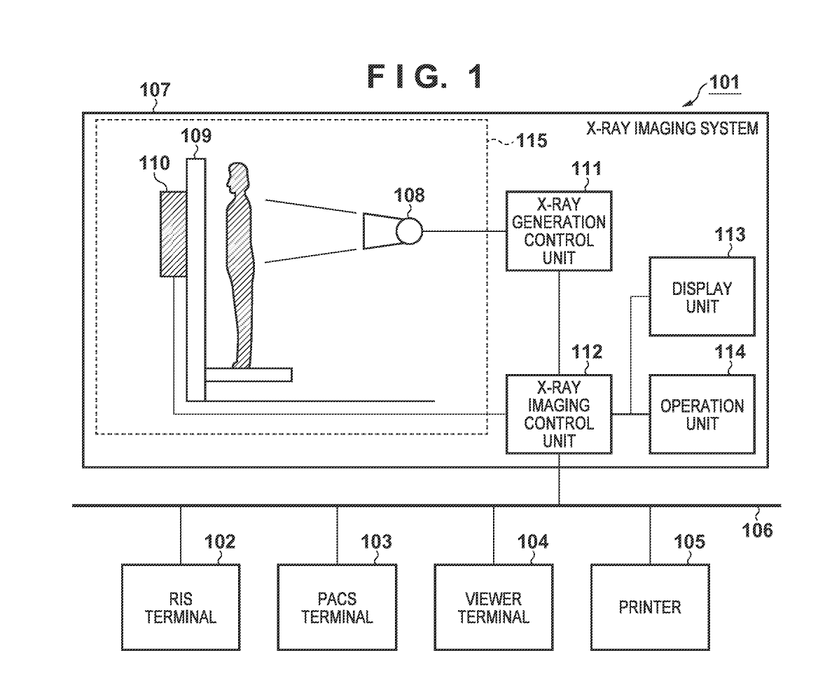

FIG. 1 shows the overall arrangement of an X-ray imaging system 101 according to an embodiment of the present invention.

The X-ray imaging system 101 includes an RIS terminal 102, a PACS terminal 103, a viewer terminal 104, a printer 105, and an X-ray imaging apparatus 107. The respective apparatuses (102 to 105 and 107) constituting the X-ray imaging system 101 are connected to each other via, for example, a communication unit 106 such as a network.

The RIS terminal 102 is an information system in the radiology department. The X-ray imaging apparatus 107 captures and obtains an X-ray digital image (to be referred to as a captured image or a radiation image hereinafter). The PACS terminal 103 stores and manages the images captured by the X-ray imaging apparatus 107. The viewer terminal 104 and the printer 105 output (display or print) diagnostic images.

The X-ray imaging apparatus 107 performs examination (imaging) based on an imaging protocol. The imaging protocol defines imaging conditions, the contents of image processing to be applied to captured images, and the like. More specifically, the protocol includes various types of parameter information for imaging operation and image processing and imaging execution information. In addition, imaging environment information (sensor type, imaging posture, and the like) is associated with the above types of information.

In this case, the X-ray imaging apparatus 107 includes an imaging unit 115, an X-ray generation control unit 111, an X-ray imaging control unit 112, a display unit 113, and an operation unit 114.

The imaging unit 115 includes an X-ray tube 108, a sensor unit 109, and a sensor 110. In this case, the X-ray tube 108 functions as a radiation generation unit and irradiates an imaging object (that is, an object) with X-rays. The X-ray generation control unit 111 controls the generation of X-rays based on the imaging protocol. More specifically, the X-ray generation control unit 111 applies a voltage to the X-ray tube 108 to generate X-rays based on imaging conditions (for example, parameters such as a tube current, tube voltage, and irradiation time) corresponding to the imaging protocol.

The sensor 110 functions as a radiation detection unit and detects the X-rays transmitted through the object. The sensor unit 109 A/D-converts electric charge corresponding to the dose of X-rays transmitted through the object detected by the sensor 110, and outputs the resultant data as a captured image to the X-ray imaging control unit 112. The X-ray imaging control unit 112 comprehensively controls information processing concerning X-ray imaging base on the imaging protocol. The X-ray imaging control unit 112 applies image processing (for example, correction processing, tone processing, and frequency processing) to, for example, a captured image. The X-ray imaging control unit 112 performs image processing by using image processing parameters corresponding to the imaging protocol at the time of imaging.

The display unit 113 is formed from, for example, a display and the like, and displays a system state and the like to the operator. The display unit 113, for example, displays the examination information received from the RIS terminal 102 (or the examination information generated by the operator). Note that “examination information” is request information which is transmitted to an RIS (Radiology Information System) in the radiology department as a request destination upon reception of an examination instruction from an HIS (Hospital Information System) terminal. The examination information includes the name of the department to which the request source belongs, an examination item, and object information.

The operation unit 114 is constituted by, for example, a keyboard, mouse, various types of buttons, and the like, and inputs an instruction from the operator into the X-ray imaging apparatus 107. The operation unit 114 inputs, for example, an image copy instruction.

### 2. Functional Arrangement of X-ray Imaging Control Unit

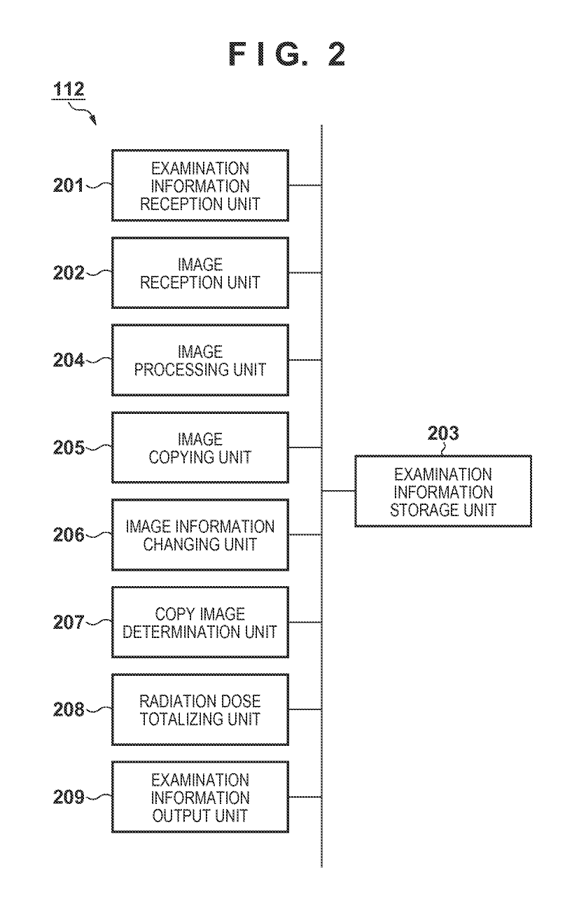

The functional arrangement of the X-ray imaging control unit 112 shown in FIG. 1 will be described next with reference to FIG. 2. As shown in FIG. 2, the X-ray imaging control unit 112 includes an examination information reception unit 201, an image reception unit 202, and an examination information storage unit 203. The X-ray imaging control unit 112 further includes an image processing unit 204, an image copying unit 205, an image information changing unit 206, a copy image determination unit 207, a radiation dose totalizing unit 208, and an examination information output unit 209.

The examination information reception unit 201 receives an input of examination information. Examination information includes the name of the department to which a request source belongs, an examination item, and object information. Examination information is input from an external system such as an HIS terminal and received via the RIS terminal 102. The image reception unit 202 receives the image captured by the X-ray imaging apparatus 107. Note that a captured image is sometimes input via a network or input via a medium such as a CD-ROM or DVD.

The examination information storage unit 203 stores the captured image input by the image reception unit 202 in association with the examination information input by the examination information reception unit 201. The image processing unit 204 applies image processing such as correction processing, tone processing, and frequency processing to captured images by using the image processing parameters included in the imaging protocol.

When the operator issues an instruction to copy the image selected with the operation unit 114, the image copying unit 205 copies the image and the image information added to the image. Note that “image information” is information indicating the attributes of a captured image. When the image copying unit 205 performs copying operation, the image information changing unit 206 automatically changes part of the image information.

The copy image determination unit 207 determines, based on the copy state (the presence/absence of a copy image attribute) included in image information, whether the image stored in the examination information storage unit 203 is a copy image or original image. The radiation dose totalizing unit 208 reads image information from the examination information storage unit 203 and calculates the total value of radiation doses applied at the time of capturing the respective images. The radiation dose totalizing unit 208 calculates the total value of radiation doses based on the determination result obtained by the copy image determination unit 207 and the image information added to captured images other than copy images. The examination information storage unit 203 stores the total value of radiation doses. The display unit 113 displays this information. The examination information output unit 209 outputs examination information and captured images to the outside (the PACS terminal 103, the viewer terminal 104, the printer 105, and the like).

### 3. Display Window of Captured Image

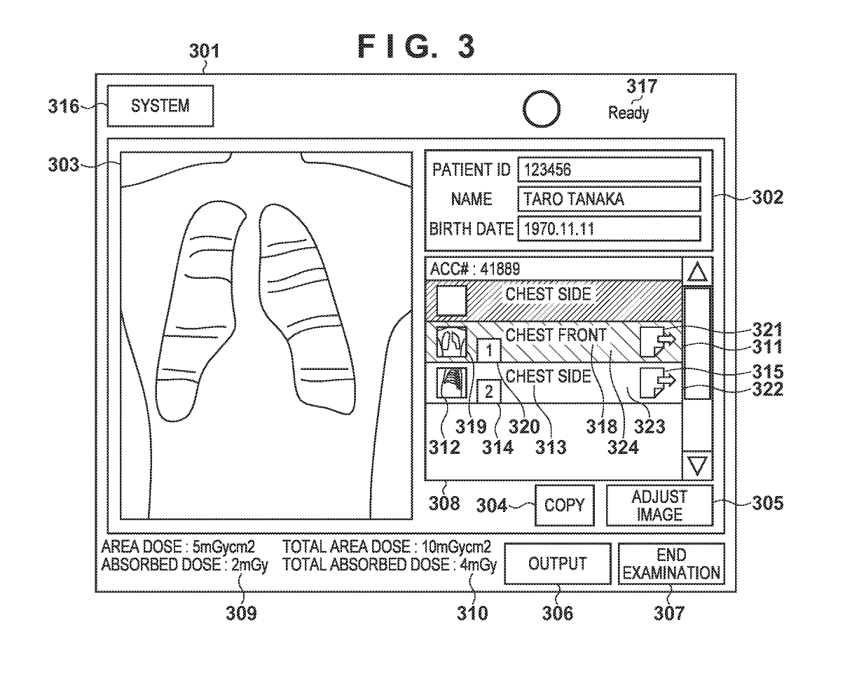

A display window displaying a captured image will be described next with reference to FIG. 3. FIG. 3 is a view showing an example of the display window for displaying a captured image, and shows a display window 301 before image copying which is displayed on the display unit 113.

As shown in FIG. 3, the display window 301 displaying the captured image includes an object information display area 302 for displaying object information, a preview image display area 303 for displaying a captured image, and an image copy button 304 for copying the preview-selected captured image.

The display window 301 also includes an image adjustment button 305 for issuing an instruction to adjust image processing parameters for the preview-selected captured image, an output button 306 for issuing an instruction to output a captured image under examination to the outside, and an examination end button 307 for issuing an instruction to end an examination. The display window 301 further includes an examination information display area 308 for displaying examination information under examination, a dose display portion 309 for displaying the radiation dose applied at the time of capturing the preview-selected image, and a total dose display portion 310 displaying the total value of the radiation doses applied throughout an examination.

The examination information display area 308 is provided with a plurality of imaging protocol buttons. Imaging protocol buttons 311 and 322 are respectively provided with thumbnail display areas 312 and 319 displaying thumbnails of captured images and name display areas 313 and 318 displaying the names of imaged regions. The examination information display area 308 is also provided with instance number display areas 314 and 320 displaying the instance numbers uniquely assigned to the respective captured images and copy attribute display portions 323 and 324 displaying identifiers (not shown in FIG. 3) indicating whether the corresponding images are copy images. The examination information display area 308 is further provided with output result display portions 315 and 321 displaying captured image output results.

Note that the imaging protocol buttons 311 and 322 each also indicate a state such as an imaging ready state, imaging preparation state, imaging completion state, or preview selected state.

In addition, the display window shown in FIG. 3 is provided with a system button 316 for issuing an instruction to transition to a system window (not shown) to change various types of system settings and a sensor state display area 317 showing the state of the sensor concerning an imaging protocol under examination. The display window shown in FIG. 3 indicates the state of the sensor in characters. However, the state of the sensor may be indicated by using a GUI, for example, changing the color of the button or displaying animation.

### 4. Procedure for Examination Processing

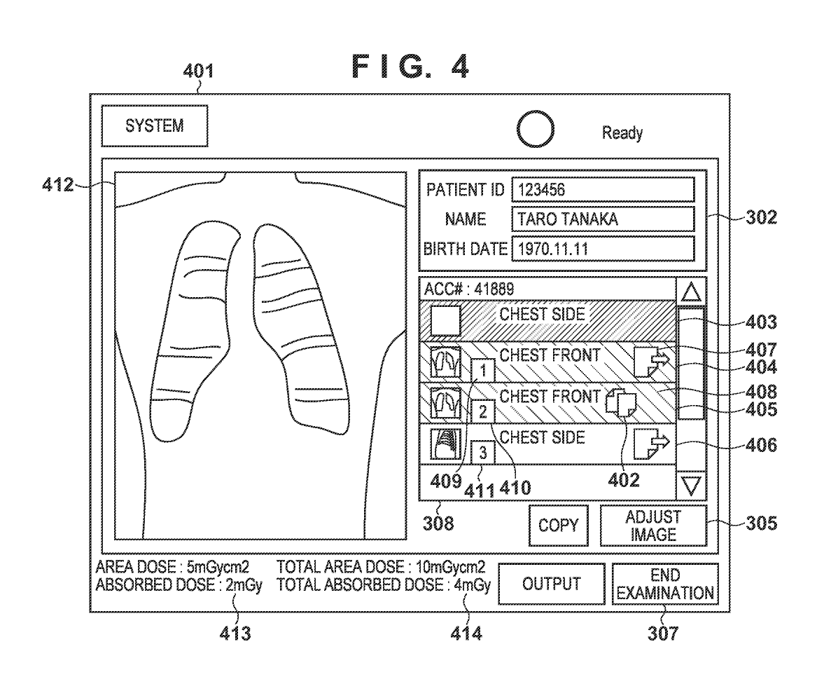

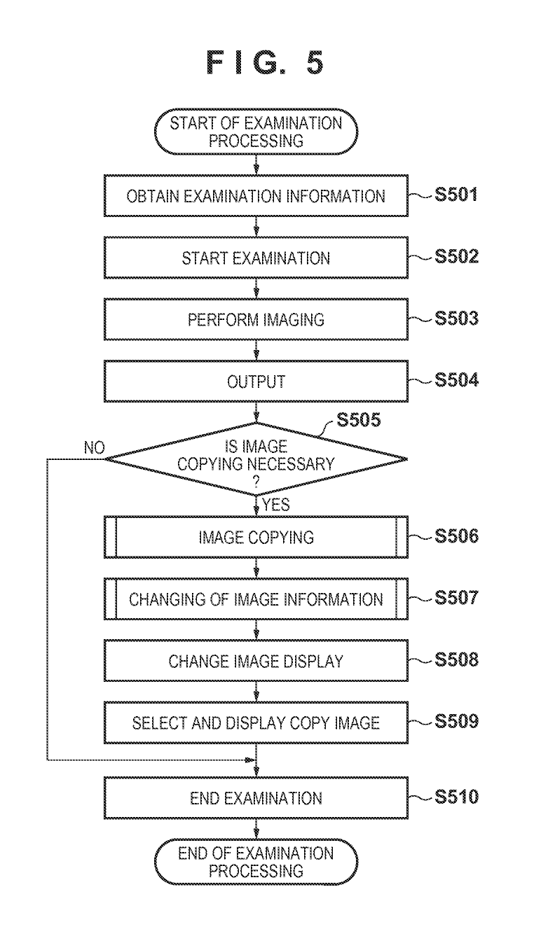

A procedure for examination processing in the X-ray imaging system 101 will be described next with reference to FIGS. 3, 4, 5, 6, and 7. FIG. 4 is a view showing an example of the display window for displaying a captured image and shows a display window 401 after image copying which is displayed on the display unit 113. FIGS. 5, 6, and 7 are flowcharts showing procedures for examination processing in the X-ray imaging system 101 and procedures for detailed processing in each step included in examination processing.

As shown in FIG. 5, in step S501, the examination information reception unit 201 receives examination information from the RIS terminal 102 (or examination information manually generated by the operator). Note that the examination information storage unit 203 stores the received examination information.

When the examination information storage unit 203 stores the examination information, the system starts an examination in step S502. The display unit 113 displays the display window 301.

In step S503, when the operator presses the irradiation button, the system performs imaging. More specifically, the X-ray tube 108 of the imaging unit 115 irradiates the object with X-rays, and the sensor 110 detects the X-rays transmitted through the object. The image reception unit 202 receives the resultant captured image. The image processing unit 204 then applies image processing such as correction processing, tone processing, and frequency processing to the received captured image. The examination information storage unit 203 stores the captured image to which the image processing has been applied in association with the examination information.

At this time, the display window 301 displays the captured image and the image information added to it. More specifically, the display window 301 displays the captured image in the preview image display area 303 and a thumbnail of the captured image in the thumbnail display area 319 of the imaging protocol button 318 corresponding to the imaging protocol used for the imaging operation. The display window 301 also displays the instance number unique to the captured image in the instance number display area 320. The display window 301 further displays the radiation dose at the time of imaging on the dose display portion 309. Note that the radiation dose totalizing unit 208 calculates the total value of the radiation doses applied throughout the examination and displays it on the total dose display portion 310.

In step S504, if the operator determines that it is necessary to output the captured image, he/she presses the output button 306 via the operation unit 114. With this operation, the examination information output unit 209 outputs the captured image and the added image information stored in the examination information storage unit 203 to the outside (the PACS terminal 103, the viewer terminal 104, the printer 105, and the like). If the output is successful, the display window 301 displays the respective output results on the captured image on the output result display portion 321. In the case in FIG. 3, for the imaging protocol buttons 311 and 322 corresponding to the captured images for which the output is successful, the display window 301 displays icons to indicate that the output is successful.

In step S505, the operator determines whether it is necessary to copy the captured image. If the operator determines that it is not necessary to copy the captured image, the process advances to step S510. If the operator determines that it is necessary to copy the captured image, the process advances to step S506.

The operator determines that it is necessary to copy the captured image, if, for example, he/she determines suspicion of pneumoconiosis upon seeing the captured image displayed in the preview image display area 303. In this case, the operator presses the imaging protocol button 311 via the operation unit 114 to display the captured image corresponding to the imaging protocol button 311 in the preview image display area 303. As is obvious from the preview-selected state of the captured image corresponding to the imaging protocol button 311, the imaging protocol button 311 is highlighted. In addition, the dose display portion 309 displays the radiation dose at the time of capturing the captured image displayed in the preview image display area 303. Assume that the operator determines suspicion of pneumoconiosis upon seeing the captured image displayed in the preview image display area 303. In this case, the operator determines that it is necessary to copy the captured image displayed in the preview image display area 303, in order to compare the captured image having undergone normal front chest image processing with the captured image having undergone pneumoconiosis image processing.

Upon determining that it is necessary to copy the captured image, the operator presses the image copy button 304 via the operation unit 114. With this operation, in step S506, the system copies the captured image displayed in the preview image display area 303.

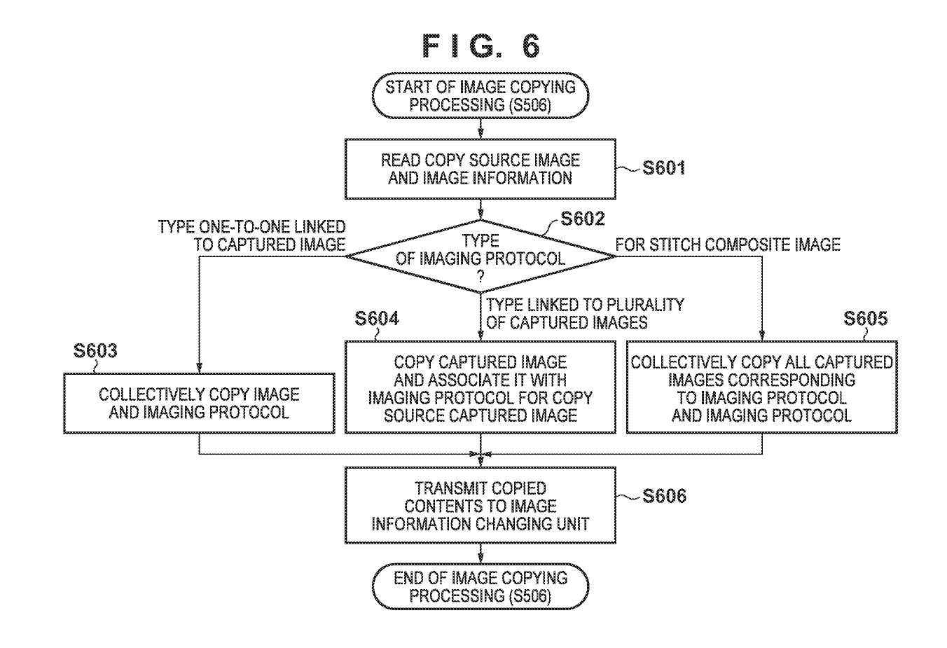

A more detailed procedure for image copying processing in step S506 will be described with reference to FIG. 6. FIG. 6 is a flowchart showing a procedure for image copying processing.

In step S601, the image copying unit 205 reads the copy source image and the image information added to the copy source image from the examination information storage unit 203. In step S602, the image copying unit 205 determines the type of imaging protocol linked to the copy source image from the read image information.

Since the imaging protocol linked to the captured image displayed in the preview image display area 303 in FIG. 3 is the type of imaging protocol one-to-one linked to the captured image, the process advances to step S603.

In step S603, the image copying unit 205 copies the copy source image, the image information added to the copy source image, and the imaging protocol linked to the copy source image. In step S606, the image copying unit 205 transmits the copy contents in the step S603 to the image information changing unit 206. With this operation, the system terminates the image copying processing in step S506. The process then advances to step S507 in FIG. 5.

Upon completion of the image copying processing by the image copying unit 205 in step S506, the image information changing unit 206 automatically performs image information changing processing in step S507.

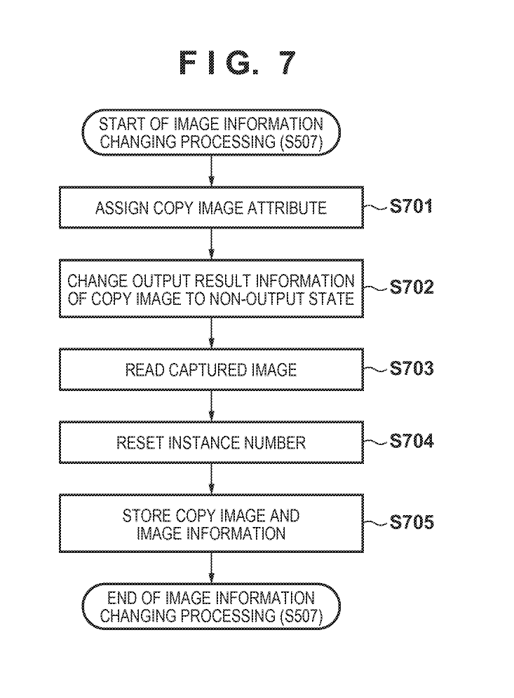

A more detailed procedure for the image information changing processing in step S507 will be described with reference to FIG. 7. FIG. 7 is a flowchart showing a procedure for the image information changing processing in step S507.

In step S701, the image information changing unit 206 assigns an attribute indicating that the corresponding image is a copy image to the image information added to the copy image received from the image copying unit 205 in step S606.

In step S702, the image information changing unit 206 changes the output result information of the image information received from the image copying unit 205 in step S606 to “non-output state”. In step S703, the image information changing unit 206 reads the captured image generated in the current examination and the added image information from the examination information storage unit 203.

In step S704, the system reassigns instance numbers to the copy image received from the image copying unit 205 in step S606 and the captured image read in step S703 so as to match their display order with the display order in the examination information display area 308. In step S705, the system stores the copy image received from the image copying unit 205 in step S606 and the image information changed by the image information changing unit 206 in the examination information storage unit 203. With this operation, the system terminates the image information changing processing in step S507. The process then returns to step S508 in FIG. 5.

In step S508, upon completion of the image information changing processing in step S507, the system automatically reflects the processing contents in steps S506 and S507 in the display unit 113. That is, the system displays the display window 401 after image copying shown in FIG. 4.

As shown in FIG. 4, the system updates the display in the examination information display area 308 after image copying. The imaging protocol button 311 in FIG. 3 is the same as an imaging protocol button 404 in FIG. 4. Likewise, the imaging protocol button 322 in FIG. 3 is the same as an imaging protocol button 406 in FIG. 4.

Since the system has copied the captured image together with the imaging protocol in step S603, the system displays, as a result, the copy image and the imaging protocol as an imaging protocol button 405. The imaging protocol button 405 is inserted after the imaging protocol button 404 corresponding to the captured image as the copy source. When updating the display in the examination information display area 308, the copy image determination unit 207 determines, by referring to the image information, whether each captured image stored in the examination information storage unit 203 is a copy image.

Since the captured image copied in step S506 has been assigned a copy image attribute in step S701, the copy image determination unit 207 determines that the image is a copy image. With this operation, the system displays a copy icon on a copy attribute display portion 402 of the imaging protocol button 405 for the copy image in the examination information display area 308.

In addition, since the output result information of the captured image copied in step S506 is changed into a non-output state in step S702, the system does not display, on an output result display portion 408, an output completion icon like that displayed on an output result display portion 407 for the copy source image.

Furthermore, since instance numbers have been reassigned in step S704, the system displays instance numbers corresponding to a display order in instance number display areas 409, 410, and 411, respectively.

In this case, the radiation dose totalizing unit 208 calculates the total value of the radiation doses applied throughout the examination and displays it on a total dose display portion 414. In this case, the radiation dose totalizing unit 208 calculates the total value by using the image information added to a captured image other than the captured image determined as a copy image by the copy image determination unit 207. For this reason, the system displays the total value of the radiation doses at the time of capturing the image except for the copy image on the total dose display portion 414.

Upon completion of the image display changing processing in step S508, the system automatically preview-selects, in step S509, the imaging protocol button 405 corresponding to the captured image copied in step S506. Upon preview-selecting the imaging protocol button 405, the system highlights the imaging protocol button 405 to indicate that it is set in the preview-selected state, as shown in FIG. 4. The system further displays the captured image corresponding to the imaging protocol button 405, that is, the captured image copied in step S506, in a preview image display area 412. This allows the operator to immediately shift to image adjusting operation for the copy image by only pressing the image adjustment button 305 via the operation unit 114.

The system displays, on a dose display portion 413, the radiation dose applied at the time of capturing the image displayed in the preview image display area 412. More specifically, the system displays the radiation dose applied at the time of capturing the copy source image of the captured image copied in step S506. For this reason, the value of the dose displayed on the total dose display portion 310 in FIG. 3 is equal to the value of the dose displayed on the dose display portion 413 in FIG. 4.

In step S510, the operator presses the examination end button 307 via the operation unit 114. With this operation, the examination information output unit 209 outputs the captured image to which image information other than the image information output in step S504 is added and the image information added to the captured image to the outside (the PACS terminal 103, the viewer terminal 104, the printer 105, and the like). The system then terminates the examination.

As described above, when the operator issues an instruction to copy a captured image one-to-one linked to an imaging protocol, the X-ray imaging system according to this embodiment discriminates this, and executes the following processing:
copying a copy source image, image information added to the copy source image, and an imaging protocol linked to the copy source image;
changing the output result information of the image information added to the copy image to the non-output state so as to match the output result;
assigning a unique instance number to each copy image;
displaying an icon indicating that the corresponding image is a copy image on the imaging protocol button so as to discriminate whether the corresponding image is the copy source image or the copy image;
automatically preview-selecting the imaging protocol button corresponding to the copy image after image copying so as to immediately perform image processing for the copy image; and
calculating the total value of the radiation doses at the time of capturing the image other than the copy image so as to match the radiation dose actually applied.

As described above, a radiation imaging system stores a radiation image, with image information indicating an attribute of the radiation image being added thereto; upon receiving an instruction to copy a radiation image, copies the radiation image and image information added to the radiation image; and changes an item, of items included in copied image information, which is unique to each of radiation images and different from each other, in accordance with a copied image, thereby changes the copied image information. The radiation imaging system switches methods of copying an imaging protocol used for obtaining a radiation image for which the copy instruction has been issued, in accordance with a relationship between the radiation image and the imaging protocol. This solves the problems unique to the radiation imaging system when copying a captured image, thereby improving the user's operability.

### Second Embodiment

The first embodiment has exemplified the processing of copying a captured image in a case in which an imaging protocol is one-to-one linked to the captured image. However, the present invention is not limited to this. The second embodiment will exemplify the processing of copying one of a plurality of captured images in a case in which the plurality of captured images are linked to one imaging protocol. Note that the overall arrangement of an X-ray imaging system according to this embodiment and the functional arrangement of an X-ray imaging control unit are the same as those in the first embodiment, and hence a description of them will be omitted.

### 1. Display Window of Captured Image

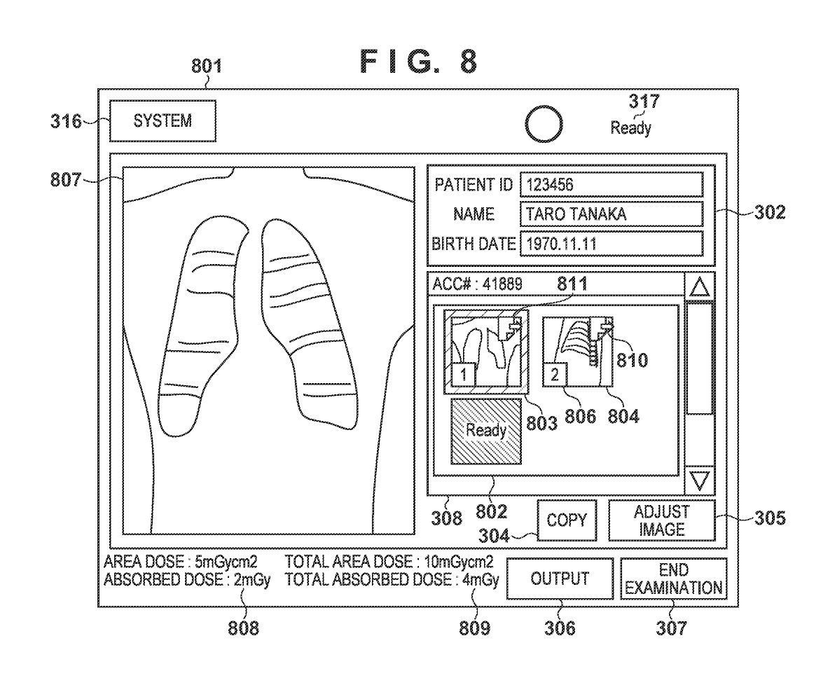

A display window which displays a plurality of captured images linked to one imaging protocol on a display unit 113 will be described first with reference to FIG. 8. FIG. 8 is a view showing an example of the display window displayed on the display unit 113 in the X-ray imaging system according to this embodiment and shows a display window 801 before image copying. Note that the same reference numerals as in FIG. 3 in the first embodiment denote the same components in FIG. 8.

As shown in FIG. 8, the display window 801 displays an imaging protocol button 802 in an examination information display area 308. On the imaging protocol button 802, the display window 801 also displays thumbnails of the images captured based on the imaging protocol in thumbnail display areas 803 and 804 in the order of imaging.

The display window 801 displays instance numbers unique to the captured images in instance number display areas 805 and 806 on the thumbnail display areas 803 and 804. The display window 801 further displays output result display portions 810 and 811 displaying icons indicating captured image output results.

The captured image in the thumbnail display area selected on the imaging protocol button 802 (the thumbnail display area 803 in the case shown in FIG. 8) is displayed in a preview image display area 807. The display window 801 displays, on a dose display portion 808, the radiation dose applied at the time of capturing the image displayed in the preview image display area 807. The display window 801 displays the total value of the radiation doses applied at the time of imaging operation using the imaging protocol on a total dose display portion 809.

### 2. Procedure for Examination Processing

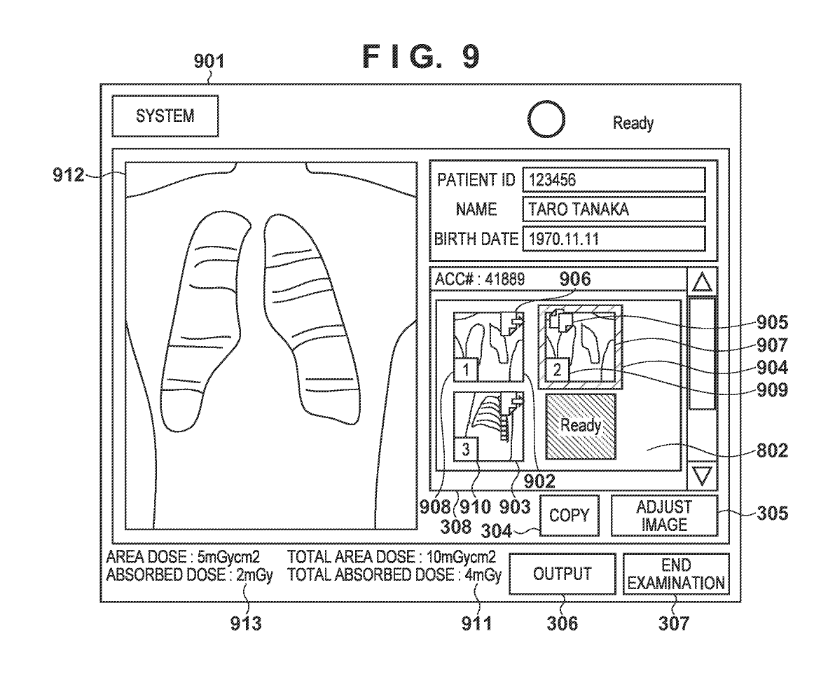

A procedure for examination processing in an X-ray imaging system 101 will be described next with reference to FIGS. 8 and 9, together with FIGS. 5 to 7.

In step S501, an examination information reception unit 201 receives examination information from an RIS terminal 102 (or examination information manually generated by the operator). Note that an examination information storage unit 203 stores the received examination information.

When the examination information storage unit 203 stores the examination information, the system starts examination in step S502. The display unit 113 displays the display window 801. Note that in this embodiment, the imaging protocol button 802 corresponding to the imaging protocol used for obtaining a plurality of captured images is displayed in the examination information display area 308.

In step S503, when the operator presses the irradiation button, the system performs imaging. More specifically, an X-ray tube 108 of an imaging unit 115 irradiates the object with X-rays, and a sensor 110 detects the X-rays transmitted through the object. An image reception unit 202 receives the resultant captured image. An image processing unit 204 then applies image processing such as correction processing, tone processing, and frequency processing to the received captured image. The examination information storage unit 203 stores the captured image to which the image processing has been applied in association with the examination information.

At this time, the display window 801 displays the captured images and the added image information. More specifically, the display window 801 displays thumbnails of the captured images in the thumbnail display areas 803 and 804 on the imaging protocol button 802 in the order of imaging. The display window 801 displays instance numbers unique to the captured images in the instance number display areas 805 and 806 on the thumbnail display areas 803 and 804. The display window 801 displays, in the preview image display area 807, the captured image in the thumbnail display area which is preview-selected and highlighted (the thumbnail display area 803 in the case shown in FIG. 8). Note that the display window 801 displays, on the dose display portion 808, the radiation dose applied at the time of capturing the image displayed in the preview image display area 807. Note also that a radiation dose totalizing unit 208 calculates the total value of the radiation doses applied throughout the examination based on the imaging protocol and displays the total value on the total dose display portion 809.

In step S504, if the operator determines that it is necessary to output the captured image, he/she presses an output button 306 via an operation unit 114. With this operation, an examination information output unit 209 outputs the captured image and the added image information stored in the examination information storage unit 203 to the outside (a PACS terminal 103, a viewer terminal 104, a printer 105, and the like). If the output is successful, the display window 801 displays the respective output results on the captured image on the output result display portions 810 and 811. In the case in FIG. 8, for the captured images for which the output is successful, the display window 801 displays icons to indicate that the output is successful.

In step S505, the operator determines whether it is necessary to copy the captured image. If the operator determines that it is not necessary to copy the captured image, the process advances to step S510. If the operator determines that it is necessary to copy the captured image, the process advances to step S506.

In step S506, the operator presses the image copy button 304 via the operation unit 114. With this operation, the system copies the captured image which is being displayed in the preview image display area 807. A detailed procedure for image copying processing in step S506 will be described with reference to FIG. 6.

In step S601, the image copying unit 205 reads the copy source image and the image information added to the copy source image from the examination information storage unit 203. In step S602, the image copying unit 205 determines the type of imaging protocol linked to the copy source image from the read image information.

Since the imaging protocol linked to the captured image displayed in the preview image display area 807 in FIG. 8 is the type of imaging protocol linked to a plurality of captured images, the process advances to step S604. In step S604, an image copying unit 205 copies the copy source image and the image information added to the copy source image, and links the copy image to the same imaging protocol as that for the copy source image. In the case shown in FIG. 8, the image copying unit 205 copies the captured image displayed as the preview-selected thumbnail in the thumbnail display area 803, and links the copy image to the imaging protocol corresponding to the imaging protocol button 802.

In step S606, the system transmits the copy contents in step S604 to an image information changing unit 206. With this operation, the system terminates the image copying processing in step S506. The flow then advances to step S507 in FIG. 5.

Upon completion of the image copying processing by the image copying unit 205 in step S506, the image information changing unit 206 automatically performs image information changing processing in step S507.

Note that since the image information changing processing in step S507 has been described in the first embodiment, a description of the processing will be omitted. Subsequently, the process advances to step S508 in FIG. 5.

In step S508, upon completion of the image information changing processing in step S507, the system automatically reflects the processing contents in steps S506 and S507 in the display unit 113. This makes it possible to display the display window 901 shown in FIG. 9 after image copying.

Reflecting the contents of processing in steps S506 and S507 will update the display in the examination information display area 308, as shown in FIG. 9. In this case, the thumbnail display area 803 in FIG. 8 is the same as a thumbnail display area 902 in FIG. 9. Likewise, the thumbnail display area 804 in FIG. 8 is the same as a thumbnail display area 903 in FIG. 9.

In step S604, the system copies the captured image and links the copy image to the same imaging protocol as that for the copy source image. As a result, the system displays a thumbnail of the copy image in a thumbnail display area 904 of the imaging protocol button 802. The thumbnail display area 904 is inserted behind the thumbnail display area 902 for the copy source image. Note that when updating the display in the examination information display area 308, a copy image determination unit 207 determines, for each captured image stored in the examination information storage unit 203, whether each image is a copy image.

Since the image information added to the captured image copied in step S506 has been assigned a copy image attribute in step S701, the copy image determination unit 207 determines that the image is a copy image. With this operation, the system displays a copy icon on a copy attribute display portion 905 of the thumbnail display area 904 for the copy image in the examination information display area 308. Note that the output result information of the captured image copied in step S506 has been changed into a “non-output state” in step S702. For this reason, the system does not display, on an output result display portion 907 in the thumbnail display area 904 for the copy image, an output completion icon like that displayed on an output result display portion 906 for the copy source image.

Furthermore, since instance numbers have been reassigned to the respective captured images in step S704, the system displays instance numbers corresponding to a display order in instance number display areas 908, 909, and 910, respectively.

Note that the radiation dose totalizing unit 208 calculates the total value of the radiation doses applied throughout the examination using the imaging protocol and displays it on a total dose display portion 911. In this case, the radiation dose totalizing unit 208 totalizes the radiation doses for a captured image other than the captured image determined as a copy image by the copy image determination unit 207. For this reason, the system displays the total value of the radiation doses for the image other than the copy image on a total dose display portion 919. For this reason, the value on the total dose display portion 809 in FIG. 8 is equal to the value on the total dose display portion 911 in FIG. 9. The process then advances to step S509.

Upon completion of the image display changing processing in step S508, the system automatically preview-selects, in step S509, the thumbnail display area 904 corresponding to the captured image copied in step S506. Upon preview-selecting the thumbnail display area 904, the system highlights the thumbnail display area 904 to indicate that it is set in the preview-selected state, as shown in FIG. 9. The system further displays the captured image corresponding to the thumbnail display area 904, that is, the captured image copied in step S506, in a preview image display area 912. This allows the operator to immediately shift to image adjusting operation for the copy image by only pressing the image adjustment button 305 via the operation unit 114.

The system displays, on a dose display portion 913, the radiation dose applied at the time of capturing the copy source image of the captured image copied in step S506.

Subsequently, the process advances to step S510. Since the processing in step S510 in this embodiment is the same as that described in the first embodiment, a description of the processing will be omitted.

As described above, according to this embodiment, when the operator issues an instruction to copy one of a plurality of captured images linked to one imaging protocol, the system discriminates this, and executes the following processing:
copying a copy source image and image information added to the copy source image and linking the resultant information to the imaging protocol to which the copy source image is linked;
changing the output result information of the image information added to the copy image to the non-output state so as to match the output result;
assigning a unique instance number to each copy image;
displaying an icon indicating that the corresponding image is a copy image on the imaging protocol button so as to discriminate whether the corresponding image is a copy source image or copy image;
automatically preview-selecting the imaging protocol button corresponding to the copy image after image copying so as to immediately perform image processing for the copy image; and
calculating the total value of the radiation doses at the time of capturing the image other than the copy image so as to match the radiation dose actually applied.

This solves the problems unique to the radiation imaging system when copying an image, thereby improving the user's operability.

### Third Embodiment

The second embodiment has exemplified the processing of copying one of a plurality of captured images linked to one imaging protocol. However, the present invention is not limited to this. The third embodiment will exemplify the processing of copying one image to which a plurality of imaging protocols are linked.

In this case, an example of one image to which a plurality of imaging protocols are linked is one stitch composite image generated by compositing a plurality of captured images to which imaging protocols are respectively linked. The processing of copying a stitch composite image will be described below.

Note that the overall arrangement of an X-ray imaging system according to this embodiment and the functional arrangement of an X-ray imaging control unit are the same as those in the first embodiment, and hence a description of them will be omitted.

### 1. Display Window of Captured Image

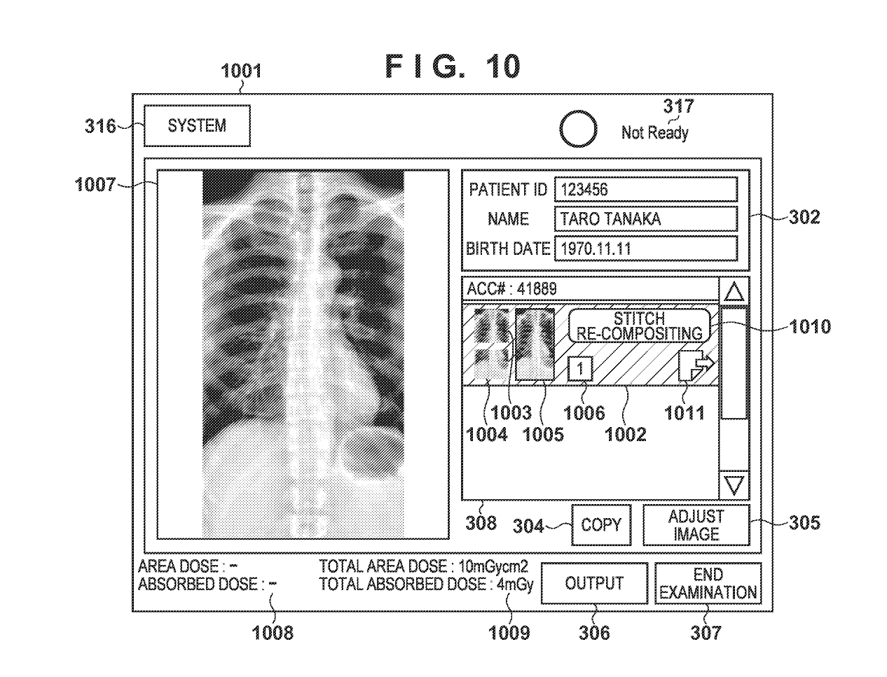

A display window displaying a stitch composite image will be described first with reference to FIG. 10. FIG. 10 is a view showing an example of the display window displayed on a display unit 113 and a display window 1001 before image copying in the X-ray imaging system according to this embodiment. Note that the same reference numerals as in FIG. 3 in the first embodiment denote the same components in FIG. 10.

As shown in FIG. 10, the display window 1001 displays an imaging protocol button 1002 in an examination information display area 308. The display window 1001 displays thumbnails of a predetermined number of captured images (partial images) for the generation of a stitch composite image in partial image thumbnail display areas 1003 and 1004 on an imaging protocol button 1002 in the order of imaging. The display window 1001 also displays a thumbnail of the stitch composite image generated by executing the stitch compositing processing of joining the predetermined number of partial images in a composite image thumbnail display area 1005.

The display window 1001 further displays an instance number unique to the stitch composite image in an instance number display area 1006 on the imaging protocol button 1002. The display window 1001 also displays an icon indicating a stitch composite image output result on an output result display portion 1011. Note that a stitch re-compositing button 1010 is a button to be used to finely adjust the compositing positions, angles, and the like of partial images.

The display window 1001 displays, in a preview image display area 1007, the image in the thumbnail display area selected on the imaging protocol button 1002 (a composite image thumbnail display area 1005 in the case shown in FIG. 10). The display window 1001 also displays, on a dose display portion 1008, the radiation dose applied at the time of capturing the image displayed in the preview image display area 1007. Note that in the case shown in FIG. 10, since the image displayed in the preview image display area 1007 is a stitch composite image and is not a captured image obtained by imaging, the display window 1001 does not display any dose on the dose display portion 1008. The display window 1001 displays the total value of the radiation doses applied at the time of capturing the respective partial images on a total dose display portion 1009.

In order to implement the functions shown in FIGS. 3, 8, and 10, an X-ray imaging control unit 112, the display unit 113, and an operation unit 114 may constitute an X-ray imaging controller. As shown in FIGS. 3, 8, and 10, the X-ray imaging controller obtains imaging information for X-ray imaging, receives the X-ray image captured in accordance with the imaging information from an imaging unit 115, displays the first image indicating the received X-ray image and imaging information corresponding to the first image and an icon for the acceptance of an operation input to instruct X-ray image copying on the display unit 113, and displays the second image based on the received X-ray image on the window in accordance with the operation input.

The system may copy a received X-ray image in accordance with an operation input. In this case, the system may obtain, as copy information, the second imaging information based on the first imaging information corresponding to the received X-ray image in accordance with an operation input. Identification information different from that of the second imaging information may be added to the first imaging information.

As shown in FIG. 3, the system may display the copy source imaging information, the copy image, the imaging information after copying, and the captured image. Alternatively, as shown FIG. 8, the system may display the copy source imaging information, the copy source image, and the captured image after copying while associating them with each other.

As shown in FIGS. 3, 8, and 10, the system may display, in specific areas on the window, imaging information which has not been used for imaging, imaging information which has been used for imaging, the first image indicating an X-ray image corresponding to the imaging information, and the second image indicating an X-ray image obtained by copying.

### 2. Procedure for Examination Processing

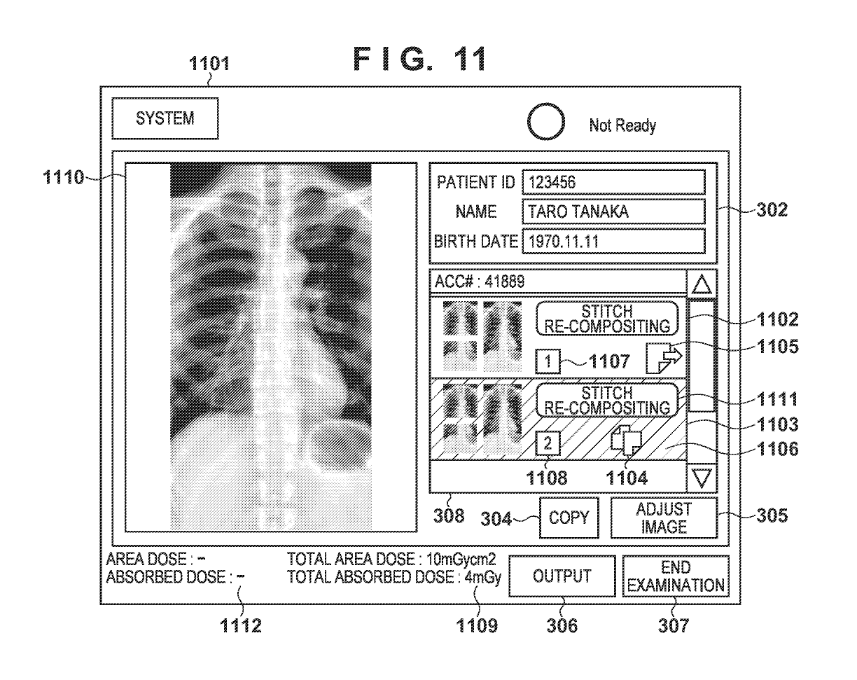

A procedure for examination processing in an X-ray imaging system 101 will be described next with reference to FIGS. 10 and 11, together with FIGS. 5 to 7.

In step S501, an examination information reception unit 201 receives examination information from an RIS terminal 102 (or manually generated by the operator). Note that an examination information storage unit 203 stores the received examination information.

When the examination information storage unit 203 stores the examination information, the system starts examination in step S502. The display unit 113 displays the display window 1001. Note that in this embodiment, the system displays the imaging protocol button 1002 corresponding to an imaging protocol for stitch imaging in the examination information display area 308 on the display window 1001.

In step S503, when the operator presses the irradiation button, the system performs imaging. More specifically, an X-ray tube 108 of the imaging unit 115 irradiates the object with X-rays, and a sensor 110 detects the X-rays transmitted through the object. An image reception unit 202 receives the resultant captured image. An image processing unit 204 then applies image processing such as correction processing, tone processing, and frequency processing to the received captured image. The examination information storage unit 203 stores the captured image to which the image processing has been applied in association with the examination information.

In this case, since the imaging protocol button 1002 is an imaging protocol button for a stitch composite image, the system captures a predetermined number of partial images used for stitch compositing processing. The system generates a stitch composite image by performing the stitch compositing processing of joining the predetermined number of partial captured images. Note that in the process of stitch compositing processing, when the system completes the stitch compositing processing upon applying various types of correction processing to the partial images, the examination information storage unit 203 stores the generated stitch composite image.

Upon generating the stitch composite image, the system displays the captured partial images, the stitch composite image, and added image information on the display window 1001. More specifically, the display window 1001 displays thumbnails of the partial images in the partial image thumbnail display areas 1003 and 1004 on the imaging protocol button 1002 in the order of imaging. The display window 1001 displays a thumbnail of the stitch composite image in the composite image thumbnail display area 1005. In addition, the display window 1001 displays an instance number unique to the stitch composite image in the instance number display area 1006.

The display window 1001 further displays, in the preview image display area 1007, the stitch composite image in the composite image thumbnail display area which is preview-selected and highlighted. The display window 1001 displays, on the dose display portion 1008, the radiation dose applied at the time of capturing the image displayed in the preview image display area 1007. In the case shown in FIG. 10, the image displayed in the preview image display area 1007 is a stitch composite image. That is, this image is not a captured image obtained by imaging, and hence no dose is displayed on the dose display portion 1008. On the other hand, a radiation dose totalizing unit 208 calculates the total value of the radiation doses applied throughout the examination, and displays the total value on the total dose display portion 1009.

In the processing in step S503, when the operator determines that it is necessary to re-composite a stitch composite image, while the imaging protocol button 1002 is displayed, the operator presses the stitch re-compositing button 1010 via the operation unit 114. With this operation, the system makes transition to stitch re-compositing processing (not shown) to allow the operator to finely correct the compositing positions and angles of partial images.

In step S504, if the operator determines that it is necessary to output the image, he/she presses an output button 306 via the operation unit 114. With this operation, an examination information output unit 209 outputs the stitch composite image, partial images, and added image information stored in the examination information storage unit 203 to the outside (a PACS terminal 103, a viewer terminal 104, a printer 105, and the like). If the output is successful, the display window 1001 displays the respective output results on the output result display portion 1011. In the case in FIG. 10, for the stitch composite image for which the output is successful, the display window 1001 displays icons to indicate that the output is successful.

In step S505, the operator determines whether it is necessary to copy the stitch composite image. If the operator determines that it is not necessary to copy the stitch composite image, the process advances to step S510. If the operator determines that it is necessary to copy the stitch composite image, the process advances to step S506.

In step S506, the operator presses the image copy button 304 via the operation unit 114. With this operation, the system copies the stitch composite image which is being displayed in the preview image display area 1007. A detailed procedure for image copying processing in step S506 will be described with reference to FIG. 6.

In step S601, an image copying unit 205 reads the stitch composite image as a copy source image, the partial images, and the image information added to the partial images from the examination information storage unit 203. In step S602, the image copying unit 205 determines the type of imaging protocol linked to the copy source image from the read image information.

Since the imaging protocol for the image displayed in the preview image display area 1007 in FIG. 10 is an imaging protocol for stitch imaging, the process advances to step S605. In step S605, the image copying unit 205 collectively copies all the partial images linked to the imaging protocol, the added image information, the stitch composite image, and the imaging protocol. More specifically, the image copying unit 205 copies a stitch composite image of the partial images in the partial image thumbnail display areas 1003 and 1004, the image information for the respective images, the imaging protocol, and the stitch composite image in the composite image thumbnail display area 1005.

In step S606, the system transmits the copy contents in step S605 to the image information changing unit 206. With this operation, the system terminates the image copying processing in step S506. The flow then advances to step S507 in FIG. 5.

Upon completion of the image copying processing by the image copying unit 205 in step S506, the image information changing unit 206 automatically performs image information changing processing in step S507. Note that since the image information changing processing in step S507 has already been described in the first embodiment, a description of the processing will be omitted. Subsequently, the process advances to step S508 in FIG. 5.

In step S508, upon completion of the image information changing processing in step S507, the system automatically reflects the processing contents in steps S506 and S507 on the display unit 113. With this operation, the system displays a display window 1101 after image copying shown in FIG. 11.

Reflecting the contents of processing in steps S506 and S507 will update the display in the examination information display area 308, as shown in FIG. 11. In this case, the imaging protocol button 1002 in FIG. 10 is the same as an imaging protocol button 1102 in FIG. 11.

In step S605, the system copies all the partial images, stitch composite image, and imaging protocol, and hence displays the copied partial images, stitch composite image, and imaging protocol as an imaging protocol button 1103.

As shown in FIG. 11, the imaging protocol button 1103 is inserted behind the imaging protocol button 1102. Note that when the display in the examination information display area 308 is to be updated, a copy image determination unit 207 determines whether the stitch composite image stored in the examination information storage unit 203 is a copy image. The copy image determination unit 207 determines the stitch composite image copied in step S506 is a copy image, because a copy image attribute is assigned to the image in step S701. For this reason, the system displays a copy icon on a copy attribute display portion 1104 of the imaging protocol button 1103 in the examination information display area 308.

In addition, the output result information of the stitch composite image copied in step S506 has been changed into the non-output state in step S702. For this reason, the system does not display, on an output result display portion 1106 of the imaging protocol button 1103 for a copy image, an output completion icon like that displayed on an output result display portion 1105 of the imaging protocol button 1102 for a copy source image.

Furthermore, since instance numbers have been reassigned to the stitch composite images in step S704, the system displays the instance numbers in instance number display areas 1107 and 1108 in the order of display.

Note that the radiation dose totalizing unit 208 calculates the total value of the radiation doses applied throughout the examination and displays the total value on a total dose display portion 1109. The radiation dose totalizing unit 208 calculates the total value with respect to images other than those determined by the copy image determination unit 207 as coy images. Therefore, the radiation dose totalizing unit 208 calculates the total value of the radiation doses for images other than copy images and displays the total value on the total dose display portion 1109. As a consequence, the total value of the doses on the total dose display portion 1009 in FIG. 10 is the same as that on the total dose display portion 1109 in FIG. 11. The process then advances to step S509.

Upon completion of the processing in step S508, the system automatically preview-selects, in step S509, the imaging protocol button 1103 corresponding to the stitch composite image copied in step S506. Upon preview-selecting the imaging protocol button 1103, the system highlights the imaging protocol button 1103 to indicate that it is set in the preview-selected state, as shown in FIG. 11. The system further displays the stitch composite image corresponding to the imaging protocol button 1103, that is, the stitch composite image copied in step S506, in a preview image display area 1110. This allows the operator to immediately shift to image adjusting operation for the copy image by only pressing the image adjustment button 305 via the operation unit 114. In addition, the operator can immediately make transition to stitch re-compositing processing (not shown) to finely correct the compositing positions and angles of partial images by only pressing a stitch re-compositing button 1111 via the operation unit 114.

The system displays, on a dose display portion 1112, the radiation dose applied at the time of imaging is displayed concerning the captured image displayed in the preview image display area 1110. The image displayed in the preview image display area 1110 in the case shown in FIG. 11 is a stitch composite image. That is, since this image is not a captured image obtained by imaging, the system displays no dose on the dose display portion 1112.

The process then advances to step S510. Note that since the processing in step S510 has already been described in the first embodiment, a description of the processing will be omitted.

As described above, when the operator issues an instruction to copy a stitch composite image, the X-ray imaging system according to this embodiment discriminates this, and executes the following processing:
copying partial images as copy source images, stitch composite image, image information added to the partial images, and imaging protocols linked to the partial images;
changing the output result information of the image information added to the copy image to the non-output state so as to match the output result;
assigning a unique instance number to each stitch composite image;
displaying an icon indicating that the corresponding image is a copy image so as to discriminate whether the corresponding image is the copy source image or the copy image;
calculating the total value of the radiation doses at the time of capturing images other than copy images so as to match the radiation dose actually applied; and
automatically preview-selecting the imaging protocol button corresponding to the copy image after image copying so as to immediately perform image processing for the copy image

This solves the problems unique to the radiation imaging system when copying a stitch composite image, thereby improving the user's operability.

### Fourth Embodiment

The first to third embodiments have exemplified the case in which each type of processing is executed on the premise that imaging has been successful in the radiation imaging system. However, the present invention is not limited to this. For example, even if the radiation imaging system has failed in imaging and performed re-imaging, the system can perform each type of processing in the same manner. This embodiment will exemplify in detail the processing to be performed in accordance with success or failure of imaging. The overall arrangement of the X-ray imaging system according to this embodiment is the same as that in the first embodiment, and hence a description of the arrangement will be omitted.

### 1. Functional Arrangement of X-ray Imaging Control Unit

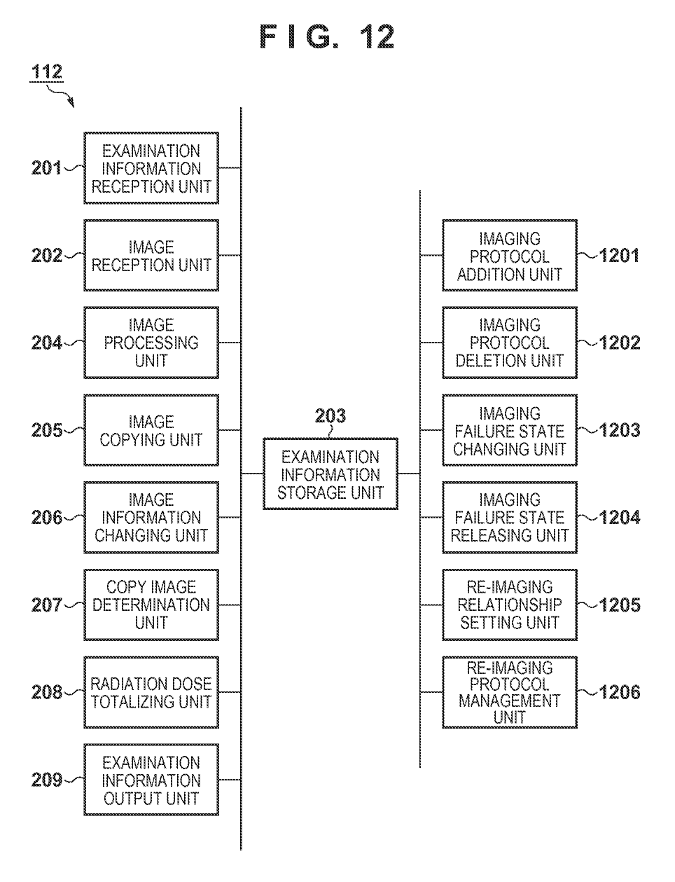

The functional arrangement of an X-ray imaging control unit 112 of an X-ray imaging system 101 according to this embodiment will be described first with reference to FIG. 12. The same reference numerals as in FIG. 2 in the first embodiment denote the same functions in FIG. 12.

Referring to FIG. 12, reference numeral 1201 denotes an imaging protocol addition unit which adds a new imaging protocol to an examination under execution; 1202, an imaging protocol deletion unit which deletes an imaging protocol which has not been used for imaging from an examination under execution; 1203, an imaging failure state changing unit which changes the success/failure state of image information added to a captured image to an imaging failure state; and 1204, an imaging failure state releasing unit which releases the imaging failure state set as a success/failure state in image information added to a captured image.

Reference numeral 1205 denotes a re-imaging relationship setting unit which sets the relationship between an imaging protocol used for imaging before re-imaging and a re-imaging protocol used for re-imaging as a re-imaging relationship; and 1206, a re-imaging protocol management unit. If there is any imaging protocol for a captured image set in a re-imaging relationship by the re-imaging relationship setting unit 1205, the re-imaging protocol management unit 1206 issues an instruction to delete the re-imaging protocol in the re-imaging relationship or an instruction to change the success/failure state of image information.

### 2. Procedure for Examination Processing

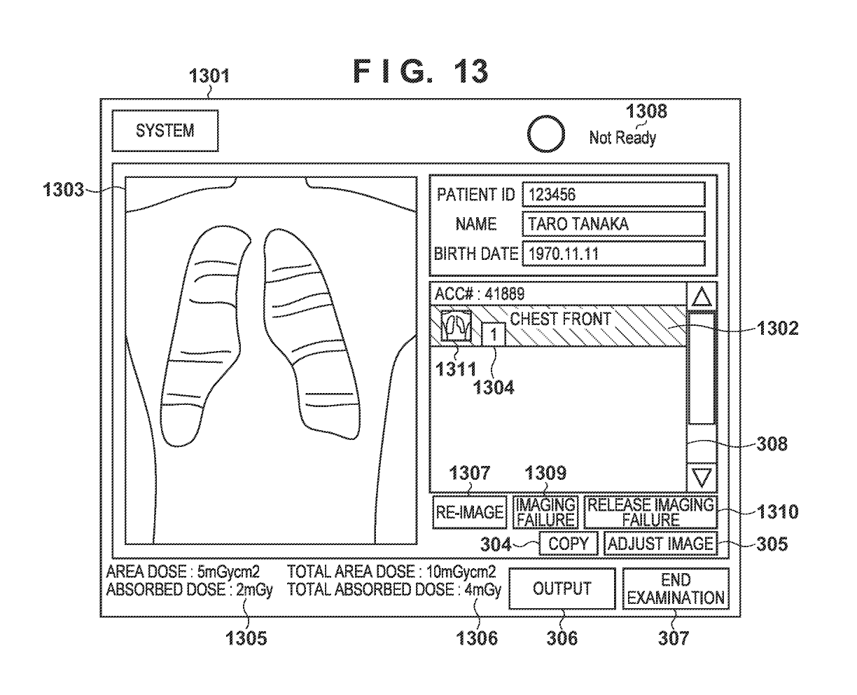

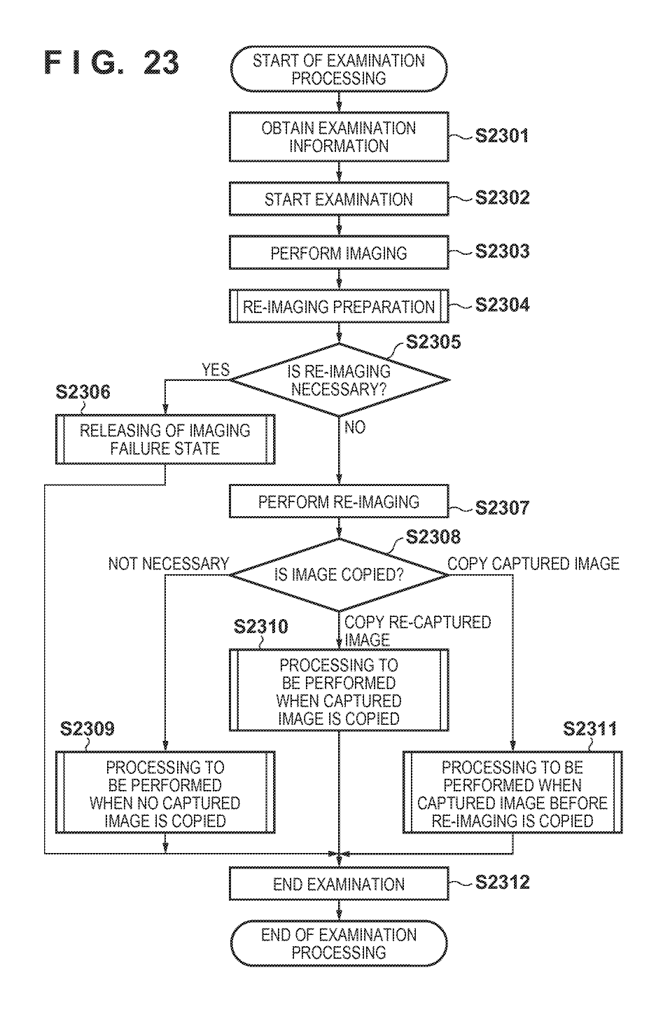

A procedure for examination processing in the X-ray imaging system 101 will be described with reference to the display windows shown in FIGS. 13 to 22, together with FIGS. 23 to 30.

As shown in FIG. 23, in step S2301, an examination information reception unit 201 receives examination information from an RIS terminal 102 (or examination information manually generated by the operator). Note that an examination information storage unit 203 stores the received examination information.

When the examination information storage unit 203 stores the examination information, the system starts examination in step S2302. A display unit 113 displays a display window 1301. Note that in this embodiment, an imaging protocol button 1302 corresponding to an imaging protocol of a type one-to-one linked to a captured image is displayed in the examination information display area 308.

In step S2303, when the operator presses the irradiation button, the system performs imaging. More specifically, an X-ray tube 108 of an imaging unit 115 irradiates the object with X-rays, and a sensor 110 detects the X-rays transmitted through the object. An image reception unit 202 receives the resultant captured image. An image processing unit 204 then applies image processing such as correction processing, tone processing, and frequency processing to the received captured image. The examination information storage unit 203 stores the captured image to which the image processing has been applied in association with the examination information.

At this time, the display window 1301 displays the captured image and the added image information. More specifically, the display window 1301 displays the captured image in a preview image display area 1303 and a thumbnail of the captured image in a thumbnail display area 1311 of the imaging protocol button 1302. The display window 1301 displays an instance number unique to the captured image in an instance number display area 1304. The display window 1301 further displays the radiation dose at the time of imaging on a dose display portion 1305. A radiation dose totalizing unit 208 calculates the total value of the radiation doses applied throughout the examination and displays the total value on a total dose display portion 1306.

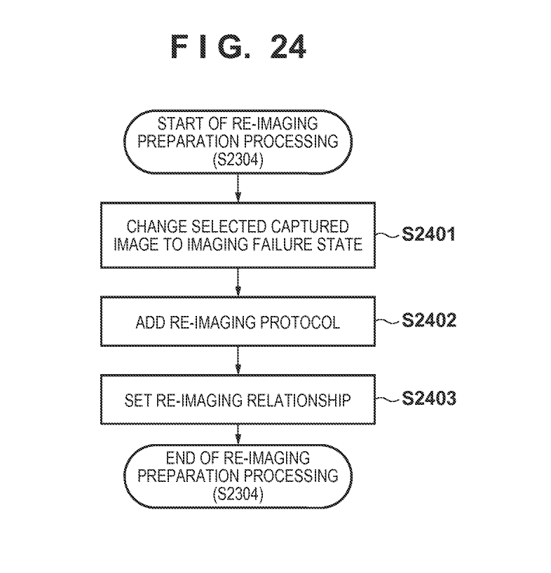

Upon checking the captured image displayed in the preview image display area 1303 and determining that re-imaging is required, the operator presses a re-imaging button 1307 via an operation unit 114 to issue an instruction to prepare for re-imaging. In step S2304, the system performs re-imaging preparation processing. A detailed procedure for re-imaging preparation processing in step S2304 will be described with reference to FIG. 24.

In step S2401, the imaging failure state changing unit 1203 sets and changes the success/failure state of image information added to the captured image displayed in the preview image display area 1303, that is, the captured image corresponding to the imaging protocol button 1302, to an imaging failure state.

In step S2402, the imaging protocol addition unit 1201 adds, as a re-imaging protocol, the same imaging protocol as that used at the time of capturing the image changed to the imaging failure state in step S2401.

In step S2403, the re-imaging relationship setting unit 1205 sets the imaging protocol for the captured image changed to the imaging failure state in step S2401 and the re-imaging protocol added in step S2402 as protocols having a re-imaging relationship. With this operation, the system terminates the re-imaging preparation in step S2304.

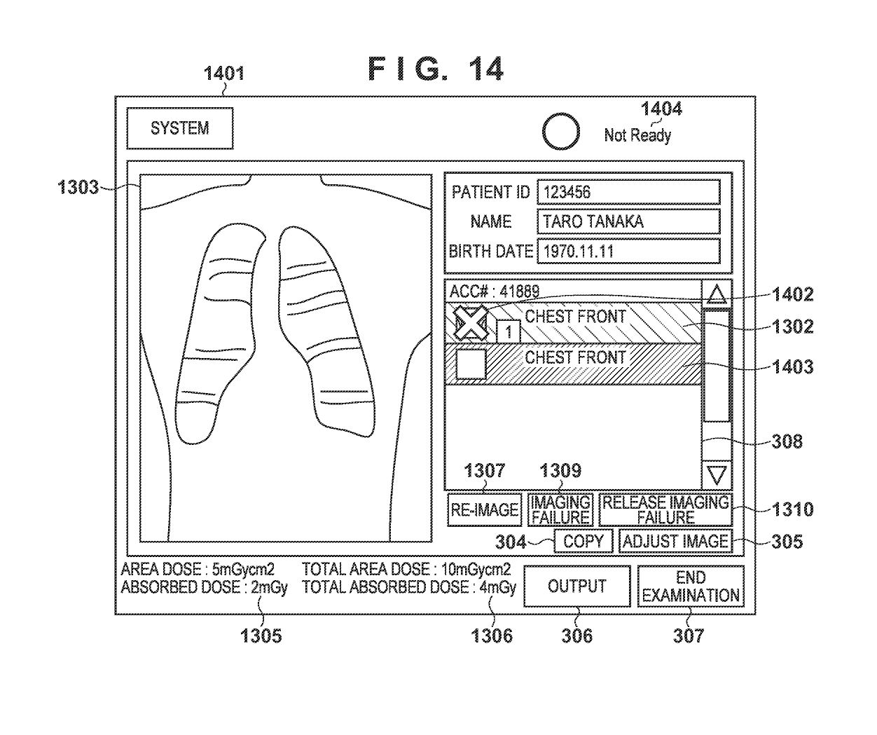

In this case, the processing contents in step S2304 are reflected in the display unit 113, and the display unit 113 displays a window 1401 shown in FIG. 14. Note that the same reference numerals as in FIG. 13 denote the same components in FIG. 14.

In step S2401, since the captured image corresponding to the imaging protocol button 1302 has been changed to the imaging failure state, the system displays an icon indicating the imaging failure state in a thumbnail display area 1402 of the imaging protocol button 1302.

In addition, the system displays a re-imaging protocol button 1403 added in step S2402 in an examination information display area 308, and highlights the re-imaging protocol button 1403 to indicate an imaging ready state. As the re-imaging protocol button 1403 is set in the imaging ready state, the system displays the imaging ready state in a sensor state display area 1404 in FIG. 14 in contrast to the unready state in a sensor state display area 1308 in FIG. 13.

In step S2305, the operator determines whether re-imaging is necessary. If the operator determines in step S2305 that he/she stops re-imaging preparation in step S2304, the process advances to step S2306. If the operator determines that he/she will execute re-imaging, the process advances to step S2307.

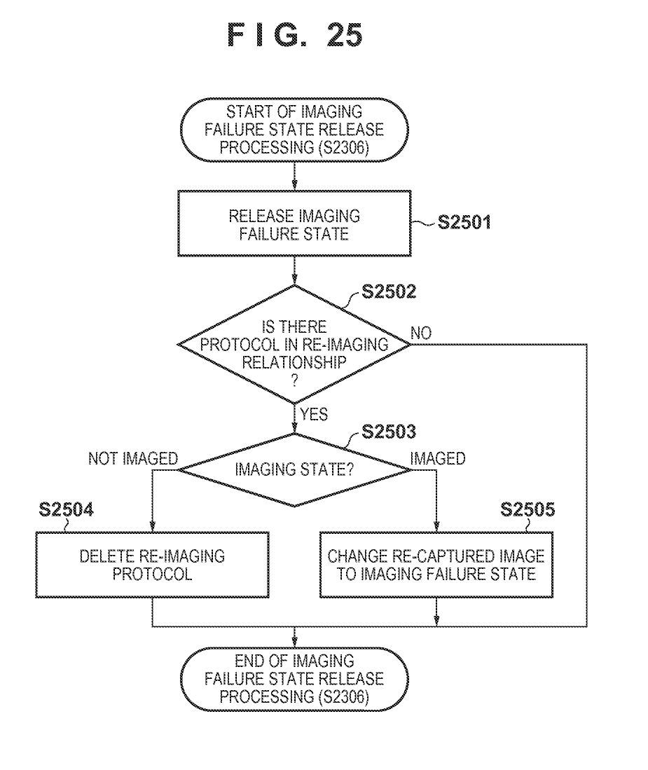

A detailed procedure for the processing to be performed in step S2306 when the operator determines that he/she stops re-imaging preparation will be described with reference to FIG. 25.

Assume that in step S2501, the operator determines, as a result of re-checking of the captured image in the preview image display area 1303, that re-imaging has not been necessary in fact. In this case, the operator presses an imaging failure release button 1310 via the operation unit 114 to issue an instruction to release the imaging failure state. With this operation, the imaging failure state releasing unit 1204 releases the imaging failure state set as the success/failure state of the image information added to the captured image corresponding to the imaging protocol button 1302.

In step S2502, the re-imaging protocol management unit 1206 determines, in accordance with the imaging failure state release processing in step S2501, whether there is any imaging protocol in a re-imaging relationship with the imaging protocol for the captured image whose imaging failure state has been released. In this case, since the re-imaging protocol corresponding to the re-imaging protocol button 1403 is set in a re-imaging relationship in step S2403, the process advances to step S2503.

In step S2503, the re-imaging protocol management unit 1206 determines the imaging state of the re-imaging protocol. In this case, since no re-imaging has been performed based on the re-imaging protocol corresponding to the re-imaging protocol button 1403, the process advances to step S2504.

In step S2504, the re-imaging protocol management unit 1206 instructs the imaging protocol deletion unit 1202 to perform deletion with respect to the re-imaging protocol button 1403. The imaging protocol deletion unit 1202 deletes the re-imaging protocol corresponding to the re-imaging protocol button 1403, and terminates the processing of stopping re-imaging preparation in step S2305. In this case, the processing contents in step S2305 are reflected in the display unit 113, and the display window 1401 returns to the display window 1301.

As indicated by the display window 1301, since the imaging failure state of the captured image corresponding to the imaging protocol button 1302 is released in step S2501, the icon indicating the imaging failure state in the thumbnail display area 1402 disappears. In addition, since the re-imaging protocol corresponding to the re-imaging protocol button 1403 is deleted in step S2504, the re-imaging protocol button 1403 is also deleted. Upon completion of the processing in step S2306, the process advances to step S2312.

A procedure for the processing to be performed when the process advances from step S2305 to step S2307 will be described next.

In step S2307, the system performs re-imaging processing by using the re-imaging protocol button 1403 in the same manner as the imaging processing in step S2303. Note that since the re-imaging processing in step S2307 is the same as step S2303 except for the contents displayed on the display unit 113, a description of the processing will be omitted.

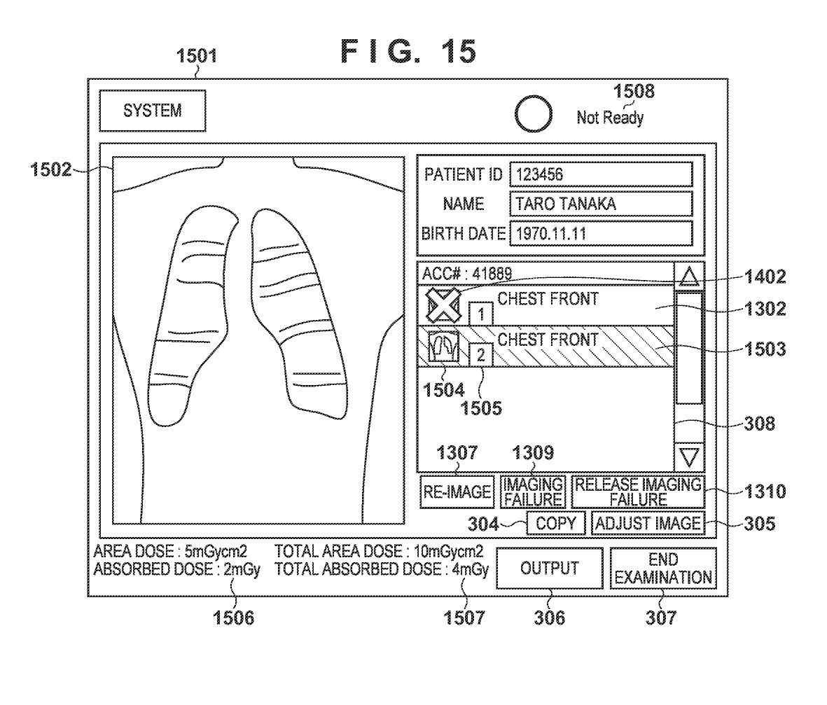

After the re-imaging processing in step S2307, the system displays the re-captured image and added image information on the display unit 113. FIG. 15 shows an example of the display window displayed on the display unit 113 after re-imaging processing in step S2307.

As shown in FIG. 15, after re-imaging processing, the system displays the re-captured image in a preview image display area 1502 and a thumbnail of the re-captured image in a thumbnail display area 1504 of a re-imaging protocol button 1503. In addition, the system displays an instance number unique to the re-captured image in an instance number display area 1505. The system also displays the radiation dose applied at the time of re-imaging on a dose display portion 1506. Furthermore, the radiation dose totalizing unit 208 calculates the total value of the radiation doses applied throughout the examination and displays the total value on a total dose display portion 1507.

Note that the re-imaging protocol button 1403 in FIG. 14 is the same as the re-imaging protocol button 1503 in FIG. 15. However, the re-captured image corresponding to the re-imaging protocol button 1503 is displayed in the preview image display area 1502, and the re-imaging protocol button 1503 is highlighted to indicate that it is in the preview-selected state.

Upon completion of re-imaging, the imaging protocol button in the imaging ready state disappears from the examination information display area 308. As a consequence, a sensor state display area 1508 returns to the unready state indication. The process advances to step S2308.

If the operator determines in step S2308 that it is not necessary to copy the re-captured image, the process advances to step S2309. If the operator determines that it is necessary to copy a re-captured image, the process advances to step S2310. If the operator determines that it is necessary to copy the captured image for which the imaging failure state is set by the imaging preparation in step S2304, the process advances to step S2311.

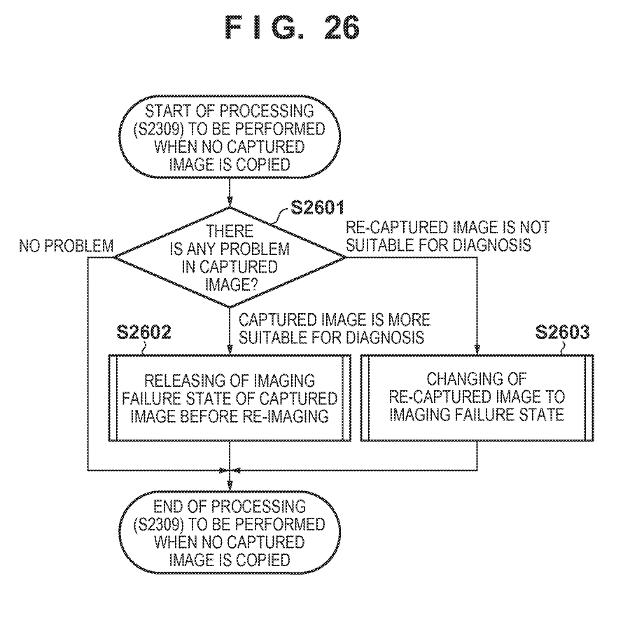

A detailed procedure for the processing in step S2309 will be described with reference to FIG. 26. If the operator determines in step S2601, upon checking the success/failure of a re-captured image, that there is no problem in the re-captured image, the system directly terminates the processing in step S2309.

If the operator determines that the captured image obtained by imaging in step S2303 is more suitable for diagnosis than the re-captured image obtained by re-imaging in step S2307, the system executes one of the following two types of processing. One type of processing is to advance to step S2602 to perform the processing of releasing the imaging failure state of the captured image obtained by imaging in step S2303, that is, the captured image before re-imaging. The other type of processing is to advance to step S2603 to perform the processing of changing the setting of the re-captured image obtained by re-imaging in step S2307 to the imaging failure state.

A detailed procedure for the processing in step S2602 will be described with reference to FIG. 25. In step S2501, the operator sets the imaging protocol button 1302 in the preview-selected state by pressing the imaging protocol button 1302 via the operation unit 114. In addition, the operator issues an instruction to release the imaging failure state by pressing the imaging failure release button 1310. With this operation, the imaging failure state releasing unit 1204 releases the imaging failure state set as the success/failure state of the image information added to the captured image obtained by imaging in step S2303, that is, the captured image corresponding to the imaging protocol button 1302.

In step S2502, upon completion of the imaging failure state release processing in step S2501, the re-imaging protocol management unit 1206 determines in step S2502 whether there is any imaging protocol having a re-capturing relationship with the imaging protocol for the captured image whose imaging failure state has been released in step S2501. In this case, since there is a re-imaging protocol corresponding to the re-imaging protocol button 1503 for which a re-imaging relationship has been set in step S2403, the process advances to step S2503.

In step S2503, the re-imaging protocol management unit 1206 determines the imaging state of the re-imaging protocol. Since the re-imaging protocol on the re-imaging protocol button 1503 has undergone re-imaging in step S2307, the process advances to step S2505.

In step S2505, the re-imaging protocol management unit 1206 issues an instruction to change the setting of the success/failure state of the re-captured image to the imaging failure state in the imaging failure state changing unit 1203. With this operation, the imaging failure state changing unit 1203 changes the setting of the success/failure state of the image information added to the re-captured image corresponding to the re-imaging protocol button 1503 to the imaging failure state. The system then terminates the processing in step S2602.

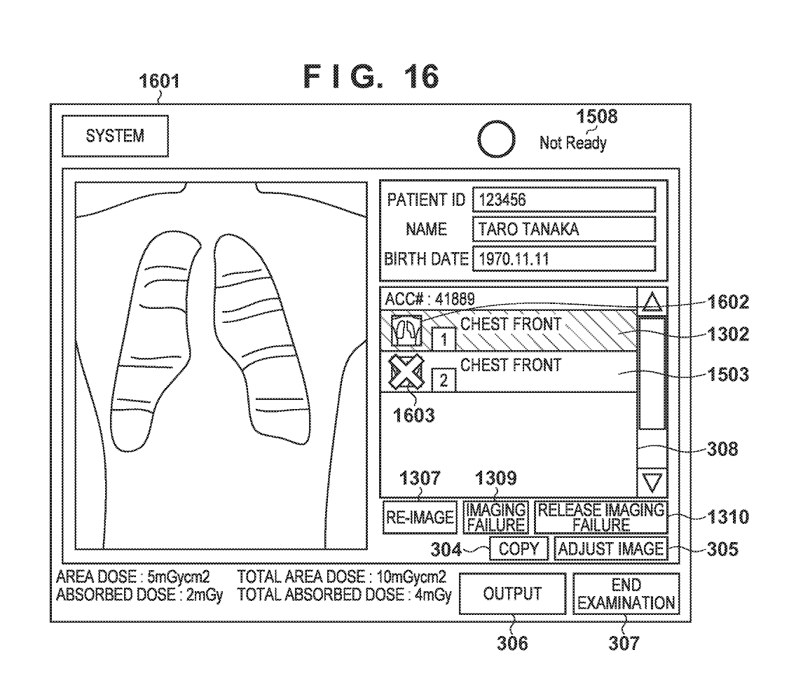

In this case, the processing contents in step S2602 are reflected in the display unit 113, and the display unit 113 displays a display window 1601 shown in FIG. 16. Note that the same reference numerals as in FIGS. 13 and 15 denote the same components in FIG. 16.

As shown in FIG. 16, since the imaging failure state of the captured image is released in step S2501, the system does not display, in a thumbnail display area 1602 in FIG. 16, the icon indicating the imaging failure state displayed in the thumbnail display area 1402 in FIGS. 14 and 15. In addition, since the setting of the re-captured image corresponding to the re-imaging protocol button 1503 has been changed to the imaging failure state in step S2505, the system displays the icon indicating the imaging failure state in a thumbnail display area 1603 of the re-imaging protocol button 1503 in FIG. 16.

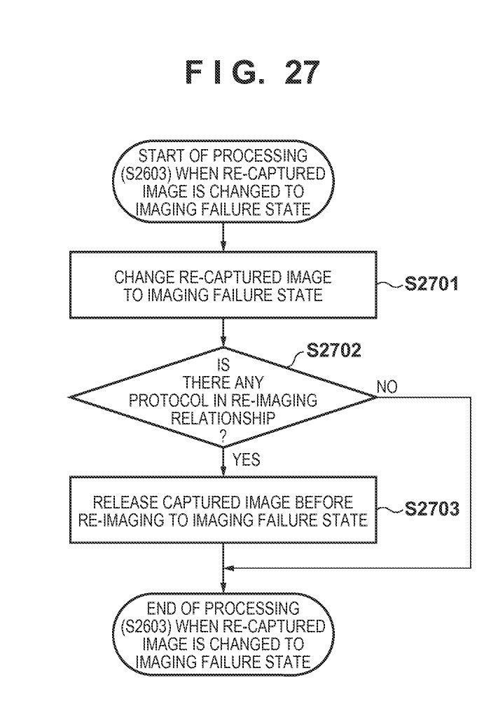

A detailed procedure for the processing in step S2603 will be described next with reference to FIG. 27. In step S2701, the operator presses the re-imaging protocol button 1503 via the operation unit 114 to set the re-imaging protocol button 1503 in the preview-selected state. The operator also presses an imaging failure button 1309 to issue an instruction to change the setting to the imaging failure state. With this operation, the imaging failure state changing unit 1203 changes the setting of the success/failure state of the image information added to the re-captured image obtained by re-imaging in step S2307 to the imaging failure state.

In step S2702, the re-imaging protocol management unit 1206 determines whether there is any imaging protocol in a re-imaging relationship with the imaging protocol for the re-captured image whose setting has been changed to the imaging failure state in step S2701. In this case, since there is an imaging protocol corresponding to the imaging protocol button 1302 which has been set in a re-imaging relationship in step S2403, the process advances to step S2703.

In step S2703, the re-imaging protocol management unit 1206 instructs the imaging failure state releasing unit 1204 to release the imaging failure state of the captured image before re-imaging, that is, the captured image corresponding to the imaging protocol button 1302. With this operation, the imaging failure state releasing unit 1204 releases the imaging failure state of the captured image corresponding to the imaging protocol button 1302. The system then terminates the processing in step S2603.

In this case, the processing contents in step S2603 are reflected in the display unit 113, and the display unit 113 displays the display window 1601 shown in FIG. 16. As shown in FIG. 16, since the re-captured image corresponding to the re-imaging protocol button 1503 has been changed to the imaging failure state in step S2701, the system displays the icon indicating the imaging failure state in the thumbnail display area 1603 of the re-imaging protocol button 1503. In addition, since the imaging failure state of the captured image corresponding to the imaging protocol button 1302 has been released in step S2703, the system does not display, in the thumbnail display area 1602, the icon indicating the imaging failure state in the thumbnail display area 1402 in FIGS. 14 and 15.

When the system completes the processing in step S2602 or S2603, the processing in step S2309 is complete. The process then advances to step S2312.

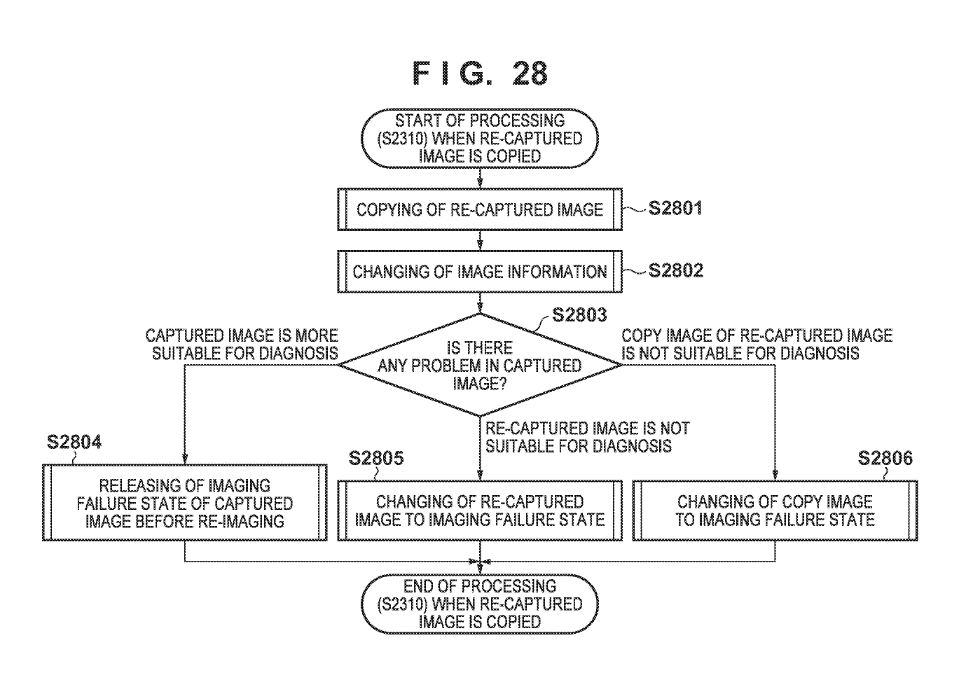

A detailed procedure for the processing in step S2310 will be described next with reference to FIG. 28. In step S2801, the operator presses an image copy button 304 via the operation unit 114 to copy the re-captured image corresponding to the re-imaging protocol button 1503. Note that a detailed procedure for the image copying processing in step S2801 is the same as that shown in FIG. 6 in the first embodiment, that is, the procedure for the processing via step S603, and hence a description of the processing will be omitted.

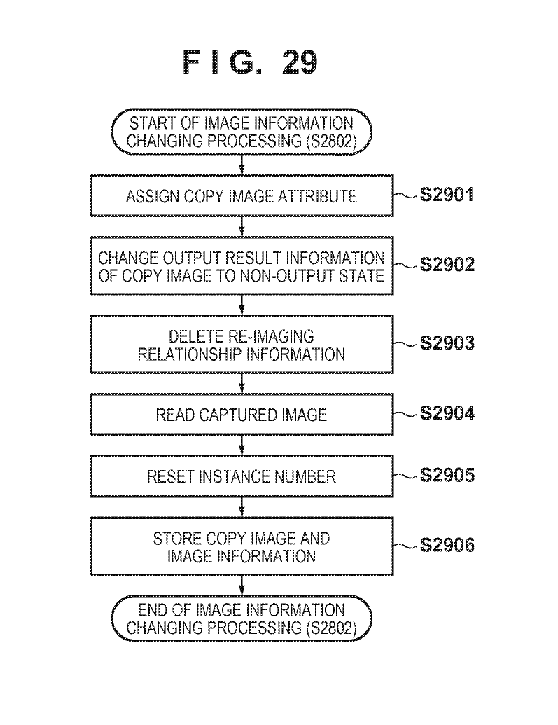

In step S2802, the system performs image information changing processing. FIG. 29 is a flowchart showing a procedure for image information changing processing. Note that the processing in steps 2901, 52902 and 52904 to 52906 in the flowchart shown in FIG. 29 is the same as that in steps S701 to S705 in FIG. 7, and hence a description of the processing will be omitted.

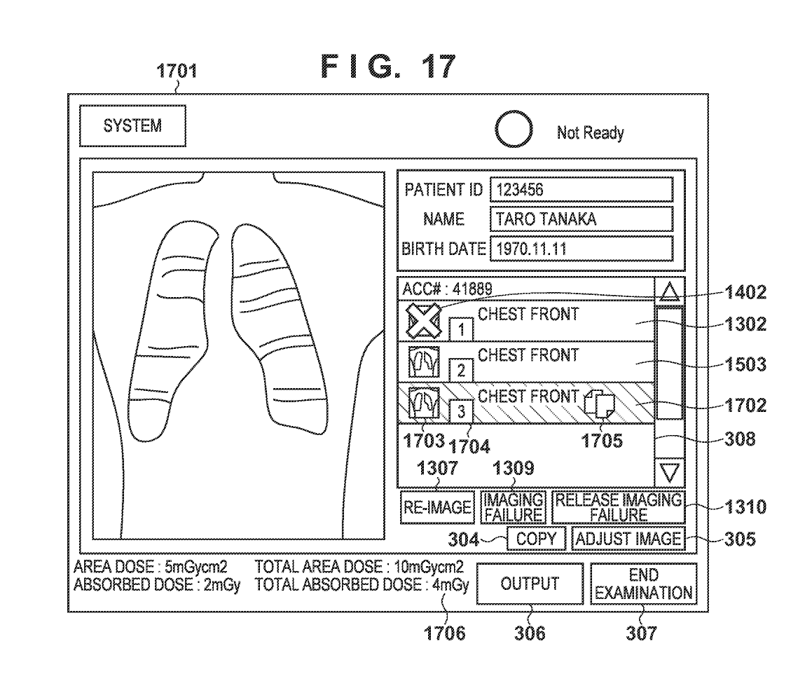

In step S2903, the image information changing unit 206 deletes the re-imaging relationship information set by the re-imaging relationship setting unit 1205 from the image information added to the copy image received from an image copying unit 205 in step S606. In this case, the processing contents in steps S2801 and S2802 are reflected in the display unit 113, and the display unit 113 displays a display window 1701 shown in FIG. 17. The same reference numerals as in FIGS. 13 and 15 denote the same components in FIG. 17.

As shown in FIG. 17, the system displays the re-captured image and re-imaging protocol copied in step S2801 as a re-imaging protocol button 1702. The system also displays a thumbnail of the copy image in a thumbnail display area 1703 and the instance number set in step S2905 in an instance number display area 1704. In addition, since a copy image determination unit 207 determines that the image is a copy image, the system displays a copy icon on a copy attribute display portion 1705 of the re-imaging protocol button 1702.

Note that the radiation dose totalizing unit 208 calculates the total value of the radiation doses applied throughout the examination, and displays the total value on a total dose display portion 1706. In this case, the radiation dose totalizing unit 208 calculates the total value of the radiation doses for captured images other than captured images determined as copy images by the copy image determination unit 207. The radiation dose totalizing unit 208 displays the calculated total value on the total dose display portion 1706. Upon completion of the processing in step S2802, the process advances to step S2803.

In step S2803, the operator checks the success/failure of each captured image. If the operator determines that there is no problem, he/she directly terminates the processing in step S2310. If the operator determines that the captured image obtained by imaging in step S2303 is more suitable for diagnosis than the re-captured image obtained by re-imaging in step S2307, the system executes one of the following three types of processing.

One type of processing is to advance to step S2804 to perform the processing of releasing the imaging failure state of the captured image obtained by imaging in step S2303, that is, the captured image before re-imaging. Another type of processing is to advance to step S2805 to perform the processing of setting the re-captured image obtained by re-imaging in step S2307 to the imaging failure state. Still another type of processing is to advance to step S2806 to perform the processing of setting the re-captured image copied in step S2801 to the imaging failure state.

Since a procedure for the processing in step S2804 is the same as that in step S2602 in FIG. 26, a description of the processing will be omitted. However, in step S2502 in FIG. 25 which explains a detailed procedure for the processing in step S2602, the system determines whether there is any imaging protocol set in a re-imaging relationship with the image captured in step S2303. In this case, however, the system executes processing different from that in step S2602 in FIG. 26. That is, since the system has deleted the re-imaging relationship information of the copy image in step S2903, the system changes only the re-captured image as a copy source, that is, the re-captured image corresponding to the re-imaging protocol button 1503, to the imaging failure state in step S2505. For this reason, the system does not change the re-captured image copied in step S2801 to the imaging failure state.

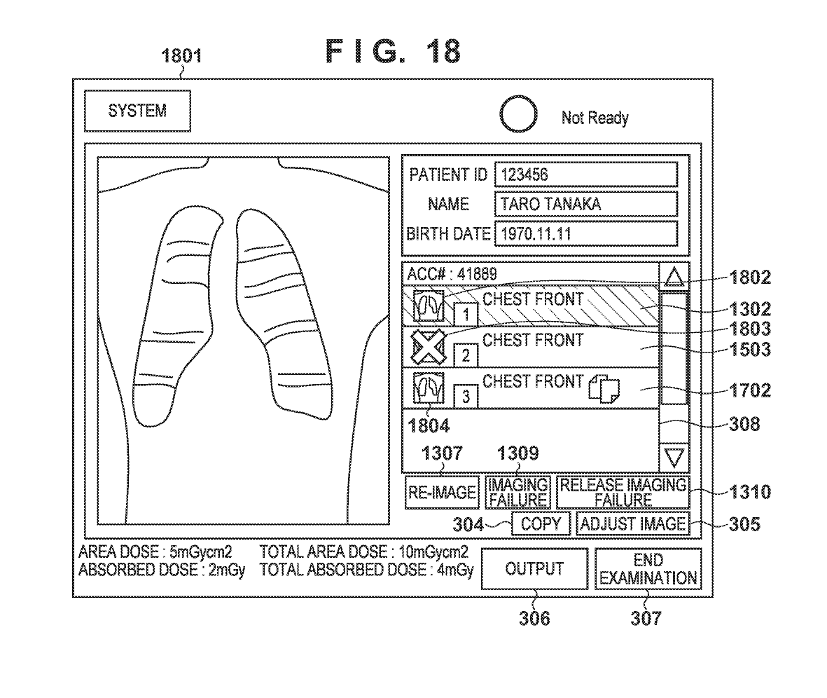

The processing contents in step S2804 are reflected in the display unit 113, and the display unit 113 displays a display window 1801 shown in FIG. 18. The same reference numerals as in FIGS. 13, 15, and 17 denote the same components in FIG. 18.

As shown in FIG. 18, since the imaging failure state of the captured image is released in step S2501, the icon indicating the imaging failure state in the thumbnail display area 1402 of the imaging protocol button 1302 disappears in a thumbnail display area 1802. In addition, since the system has changed the re-captured image corresponding to the re-imaging protocol button 1503 to the imaging failure state in step S2505, the system displays an imaging failure state icon in a thumbnail display area 1803 of the re-imaging protocol button 1503.

On the other hand, the re-captured image corresponding to the re-imaging protocol button 1702 is a copy image of the re-captured image corresponding to the re-imaging protocol button 1503. However, since the system has deleted the re-imaging relationship information in step S2903, the system does not change the success/failure state to the imaging failure state in step S2505. For this reason, the system does not display an imaging failure state icon in a thumbnail display area 1804 of the re-imaging protocol button 1702.

Step S2805 will be described next. A procedure for the processing in step S2805 is the same as that in step S2603 in FIG. 26. The processing contents in step S2805 are reflected in the display unit 113, and the display unit 113 displays the display window 1801 shown in FIG. 18. Note that the same reference numerals as in FIGS. 13, 15, and 17 denote the same components in FIG. 18.

As shown in FIG. 18, since the system has changed the re-captured image corresponding to the re-imaging protocol button 1503 to the imaging failure state in step S2701, the system displays an imaging failure state icon in the thumbnail display area 1803 of the re-imaging protocol button 1503. In addition, since the system has released the imaging failure state of the re-captured image in step S2703, the imaging failure state icon in the thumbnail display area 1402 of the imaging protocol button 1302 disappears in the thumbnail display area 1802.

The procedure for the processing in step S2805 has no influence on the re-captured image corresponding to the re-imaging protocol button 1702. For this reason, the system does not display the imaging failure state icon in the thumbnail display area 1804 of the re-imaging protocol button 1702 without changing to the imaging failure state.

A detailed procedure for processing in step S2806 will be described next with reference to FIG. 27. In step S2701, the operator presses the re-imaging protocol button 1702 via the operation unit 114 to set the re-imaging protocol button 1702 in the preview-selected state. In addition, the operator presses the imaging failure button 1309 to issue an instruction to change the imaging failure state. With this operation, the imaging failure state changing unit 1203 changes the setting of the re-captured image copied in step S2801, that is, the re-captured image corresponding to the re-imaging protocol button 1702, to the imaging failure state.

In step S2702, the re-imaging protocol management unit 1206 determines whether there is any imaging protocol in a re-imaging relationship with the imaging protocol for the re-captured image whose success/failure state has been changed to the imaging failure state in step S2701.

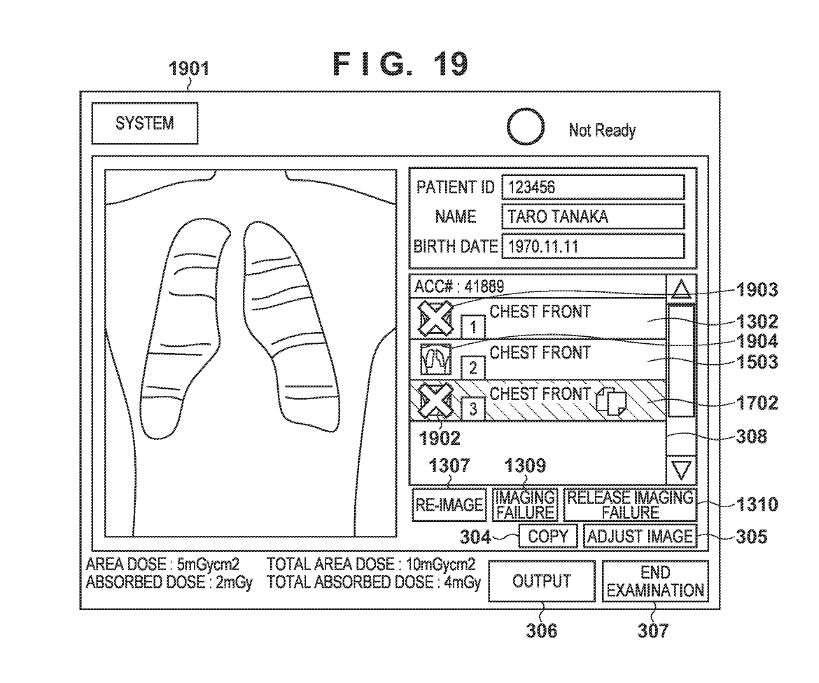

Since the system has deleted the re-imaging relationship information of the re-captured image corresponding to the re-imaging protocol button 1702 in step S2903, the re-imaging protocol management unit 1206 determines in step S2702 that there is no imaging protocol in a re-imaging relationship. The system then terminates the processing in step S2806. In this case, the processing contents in step S2806 are reflected in the display unit 113, and the display unit 113 displays a display window 1901 shown in FIG. 19. Note that the same reference numerals as in FIGS. 13, 15, and 17 denote the same components in FIG. 19.

As shown in FIG. 19, since the system has changed the setting of the re-captured image corresponding to the re-imaging protocol button 1702 to the imaging failure state in step S2701, the system displays an imaging failure state icon in a thumbnail display area 1902. In contrast, since the procedure for the processing in step S2806 has no influence on the captured image corresponding to the imaging protocol button 1302 and the re-captured image corresponding to the re-imaging protocol button 1503, the system does not change the success/failure state of each image. Therefore, the displayed or disappearing states of the imaging failure state icons in a thumbnail display area 1903 of the imaging protocol button 1302 and in a thumbnail display area 1904 of the re-imaging protocol button 1503 are the same as those on the display window 1701 shown in FIG. 17.

When the processing in steps S2804, S2805, and S2806 is complete, the processing in step S2310 is complete. The process then advances to step S2312.

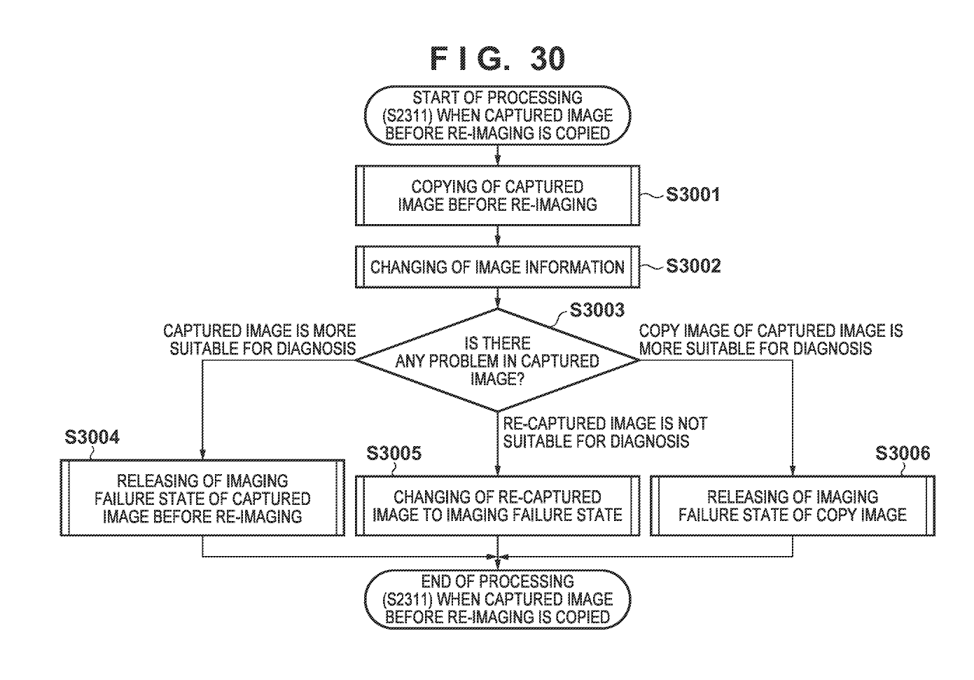

A detailed procedure for processing in step S2311 will be described next with reference to FIG. 30. In step S3001, the operator presses the imaging protocol button 1302 via the operation unit 114 to set the imaging protocol button 1302 in the preview-selected state. The operator further presses the image copy button 304 to copy the captured image corresponding to the imaging protocol button 1302. The detailed procedure for the image copying processing in step S3001 is the same as that shown in FIG. 6 in the first embodiment described above, that is, that through step S603, and hence a description of the processing will be omitted. In addition, since a detailed procedure for processing in step S3002 is the same as that in step S2802, a description of the processing will be omitted.

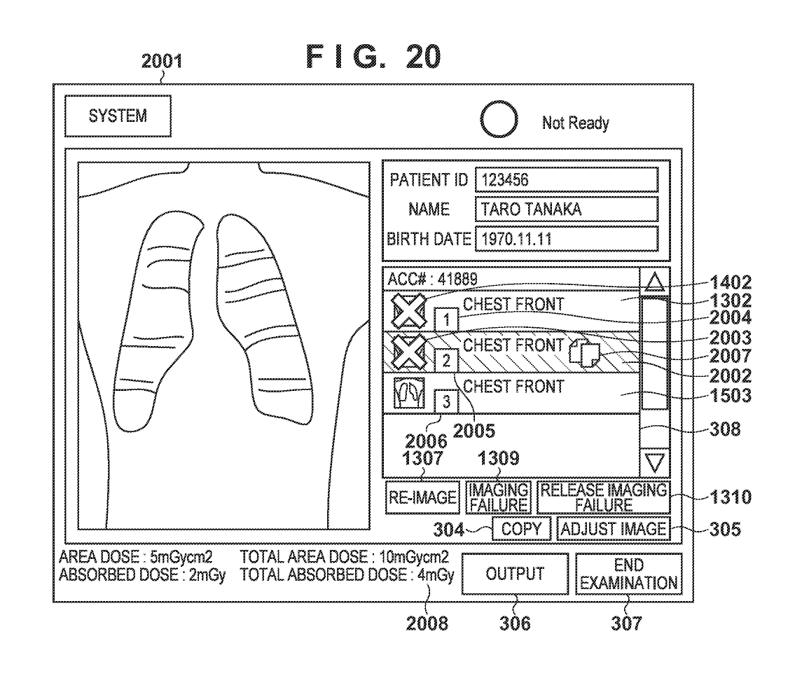

The processing contents in steps S3001 and S3002 are reflected in the display unit 113, and the display unit 113 displays a display window 2001 shown in FIG. 20. The same reference numerals as in FIGS. 13, 14, and 15 denote the same components in FIG. 20.

As shown in FIG. 20, the system displays the captured image and imaging protocol copied in step S3001 as an imaging protocol button 2002. The system also displays a thumbnail of the copied image in a thumbnail display area 2003. Since the copy source image of the captured image corresponding to the imaging protocol button 2002 is a captured image in the imaging failure state, the system also copies the imaging failure state when copying the captured image. For this reason, the system displays the imaging failure state icon in the thumbnail display area 2003.

The system displays the instance numbers reset in step S2905 in instance number display areas 2004, 2005, and 2006 on the imaging protocol buttons 1302 and 2002 and the re-imaging protocol button 1503. Since a copy image determination unit 207 determines the copy image as a copy image, the system displays a copy icon on a copy attribute display portion 2007 of the imaging protocol button 2002.

The radiation dose totalizing unit 208 calculates the total value of the radiation doses applied throughout the examination and displays the total value on a total dose display portion 2008. Note that the radiation dose totalizing unit 208 calculates the radiation doses for captured images other than captured images determined by the copy image determination unit 207 as copy images as a total value. For this reason, the radiation dose totalizing unit 208 totalizes the radiation doses for images other than copy images and displays the total value on the total dose display portion 2008. Upon completion of the processing in step S3002, the process advances to step S3003.

In step S3003, the operator checks the success/failure of each captured image. If the operator determines that there is no problem, he/she directly terminates the processing in step S2311.

If the operator determines that the captured image obtained by imaging in step S2303 is more suitable for diagnosis than the re-captured image obtained by re-imaging in step S2307, the system executes one of the following three types of processing.

One type of processing is to advance to step S3004 to perform the processing of releasing the imaging failure state of the captured image obtained by imaging in step S2303, that is, the captured image before re-imaging. Another type of processing is to advance to step S3005 to perform the processing of setting the re-captured image obtained by re-imaging in step S2307 to the imaging failure state. Still another type of processing is to advance to step S3006 to perform the processing of setting the captured image copied in step S3001 to the imaging failure state.

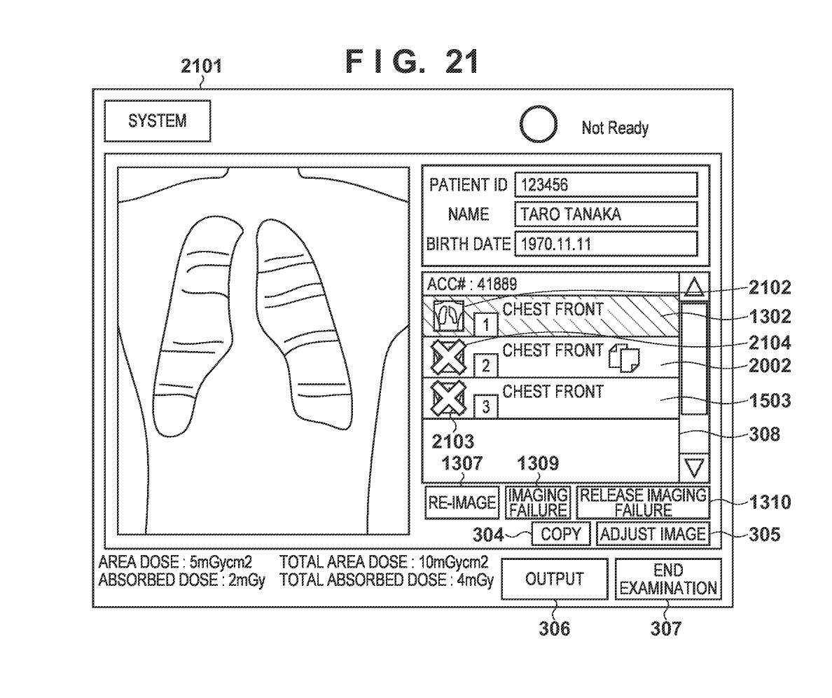

Since a procedure for the processing in step S3004 is the same as that in step S2602 in FIG. 26, a description of the processing will be omitted. Note that the processing contents in step S3004 are reflected in the display unit 113, and the display unit 113 displays a display window 2101 shown in FIG. 21. The same reference numerals as in FIGS. 13, 15, and 20 denote the same components in FIG. 21.

As shown in FIG. 21, since the system has released the imaging failure state of the captured image in step S2501, the system does not display, in a thumbnail display area 2102, the imaging failure state icon in the thumbnail display area 1402 of the imaging protocol button 1302. In addition, since the system has changed the setting of the re-captured image corresponding to the re-imaging protocol button 1503 to the imaging failure state in step S2505, the system displays the imaging failure state icon in a thumbnail display area 2103 of the re-imaging protocol button 1503.

Although the captured image corresponding to the imaging protocol button 2002 is a copy image of the captured image corresponding to the imaging protocol button 1302, the processing in step S3004 has no influence on the image. Therefore, the imaging failure state is not released. As a consequence, a thumbnail display area 2104 of the imaging protocol button 2002 is the same as the thumbnail display area 2003 in FIG. 20.

Since a procedure for processing in step S3005 is the same as that in step S2603 in FIG. 26, a description of the processing will be omitted. In step S2702 in FIG. 27 for explaining a detailed procedure for the processing in step S2603, the system determines whether there is any an imaging protocol in a re-imaging relationship with the imaging protocol for the image captured in step S2307. In this case, however, the system executes processing different from that in step S2603 in FIG. 26.

That is, since the system has deleted the re-imaging relationship information of the copy image in step S2903, the system releases only the imaging failure state of the captured image before re-imaging as a copy source, that is, the captured image corresponding to the imaging protocol button 1302 in step S2703. For this reason, the system does not release the imaging failure state of the captured image copied in step S3001. The processing contents in step S3005 are reflected in the display unit 113, and the display unit 113 displays the display window 2101 shown in FIG. 21. Note that the same reference numerals as in FIGS. 13, 15, and 20 denote the same components in FIG. 21.

As shown in FIG. 21, since the system has changed the setting of the re-captured image to the imaging failure state in step S2701, the system displays the imaging failure state icon in the thumbnail display area 2103 of the re-imaging protocol button 1503. In addition, since the system has released the imaging failure state of the captured image in step S2703, the system does not display, in the thumbnail display area 2102 of the imaging protocol button 1302, the imaging failure state icon in the thumbnail display area 1402 of the imaging protocol button 1302.

Although the captured image corresponding to the imaging protocol button 2002 is a copy of the captured image corresponding to the imaging protocol button 1302, since the system has released the re-imaging relationship information in step S2903, the system does not release the imaging failure state in step S2703. As a consequence, the thumbnail display area 2104 of the imaging protocol button 2002 is the same as the thumbnail display area 2003 in FIG. 20.

A detailed procedure for the processing in step S3006 will be described with reference to FIG. 25. In step S2501, the operator presses the imaging protocol button 2002 via the operation unit 114 to set the imaging protocol button 2002 in the preview-selected state. The operator further presses the imaging failure release button 1310 to issue an instruction to release the imaging failure state. With this operation, the imaging failure state releasing unit 1204 releases the imaging failure state of the captured image copied in step S3001, that is, the captured image corresponding to the imaging protocol button 2002.

In step S2502, the re-imaging protocol management unit 1206 determines whether there is any imaging protocol in a re-imaging relationship with the imaging protocol for the captured image whose imaging failure state has been canceled in step S2501. Since the system has deleted the re-imaging relationship information of the captured image corresponding to the imaging protocol button 2002 in step S2903, the system determines that there is no imaging protocol in re-imaging relationship, and terminates the processing in step S3006.

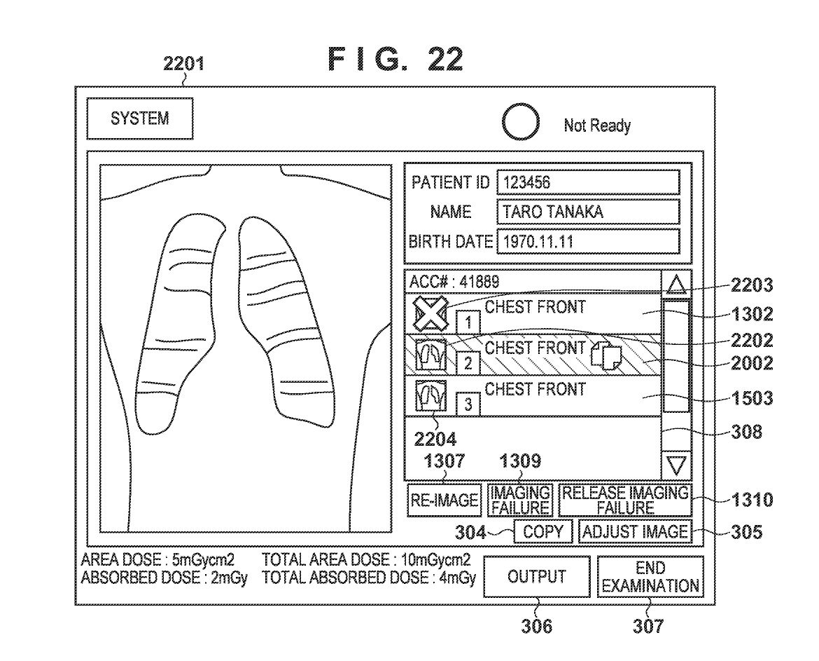

In this case, the processing contents in step S3006 are reflected in the display unit 113, and the display unit 113 displays a display window 2201 shown in FIG. 22. The same reference numerals as in FIGS. 13, 15, and 20 denote the same components in FIG. 22.

As shown in FIG. 22, since the system has released the imaging failure state of the captured image in step S2501, the system does not display, in a thumbnail display area 2202 in FIG. 22, the imaging failure state icon in the thumbnail display area 2003 of the imaging protocol button 2002. In contrast, since the procedure for the processing in step S3006 has no influence on the captured image corresponding to the imaging protocol button 1302 and the re-captured image corresponding to the re-imaging protocol button 1503, the system does not change the success/failure state of each image. Therefore, the displayed or disappearing states of the imaging failure state icons on a thumbnail display area 2203 of the imaging protocol button 1302 and on a thumbnail display area 2204 of the re-imaging protocol button 1503 are the same as those shown in FIG. 20.

When the processing in steps S3004, S3005, and S3006 is complete, the processing in step S2311 is complete. The process then advances to step S2312. Note that since the processing in step S2312 is the same as that in step S510 in the first embodiment, a description of the processing will be omitted.

As has been described above, in this embodiment, when copying a captured image before re-imaging or a re-captured image obtained by re-imaging, the system executes the following processing:
setting a success/failure state for each captured image without synchronism with changing of the success/failure state of the copy source image; and
setting a success/failure state for the copy source image without synchronism with changing of the success/failure state of each copy image.

This solves the problems unique to the radiation imaging system when copying a captured image before re-imaging or a re-captured image obtained by re-imaging upon performing re-imaging, thereby improving the user's operability.

### Other Embodiments

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (for example, computer-readable storage medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-232335 filed on Oct. 19, 2012, which is hereby incorporated by reference herein in its entirety.

What is claimed is:

1. A radiation imaging system for imaging an object to obtain a radiation image based on an imaging protocol, the system comprising:
a storage unit configured to store a radiation image, with image information indicating an attribute of the radiation image being added thereto;
a copying unit configured to, upon receiving an instruction to copy a radiation image stored in said storage unit, copy the radiation image and image information added to the radiation image; and
a changing unit configured to change an item, of items included in copied image information copied by said copying unit, which is unique to each of radiation images and different from each other, in accordance with a copied image, thereby changing the copied image information,
wherein said copying unit switches methods of copying an imaging protocol used for obtaining a radiation image for which the copy instruction has been issued, in accordance with a relationship between the radiation image and the imaging protocol.

2. The system according to claim 1, wherein said copying unit copies an imaging protocol used for obtaining a radiation image for which the copy instruction has been issued if the radiation image is one-to-one linked to the imaging protocol.

3. The system according to claim 1, wherein if a plurality of radiation images including a radiation image for which the copy instruction has been issued are linked to an imaging protocol used for obtaining the radiation images, said copying unit links the copied image to the imaging protocol.

4. The system according to claim 1, wherein if a copy instruction is issued for a stitch composite image obtained by compositing a plurality of partial images, said copy unit copies the plurality of partial images which generate the stitch composite image and imaging protocols respectively linked to the plurality of partial images when copying the stitch composite image.

5. The system according to claim 1, wherein items of the image information which are changed by said changing unit include at least an output state indicating whether the radiation image to which the image information is added is output to an outside and a copy state indicating whether an image is a copy image.

6. The system according to claim 1, further comprising:
an associating unit configured to, when a radiation image is generated by imaging an object based on an imaging protocol and an another radiation image is then generated by re-imaging, associate the imaging protocol used for obtaining the radiation image with a re-imaging protocol used for obtaining the another radiation image; and
a setting changing unit configured to change setting of a success/failure state which is included in the image information and indicates a success/failure of imaging or re-imaging,
wherein when said setting changing unit changes setting of a success/failure state included in image information added to a radiation image, said associating unit changes setting of a success/failure state included in image information added to an another radiation image generated by using a re-imaging protocol associated with an imaging protocol used for obtaining the radiation image, and
when said setting changing unit changes setting of a success/failure state included in image information added to an another radiation image, said associating unit changes setting of a success/failure state included in image information added to a radiation image generated by using an imaging protocol associated with an imaging protocol used for obtaining the another radiation image.

7. The system according to claim 5, further comprising a display unit configured to display a radiation image stored in said storage unit,
wherein items of the image information which are changed by said changing unit include an instance number assigned in a display order to the radiation image displayed by said display unit.

8. The system according to claim 7, wherein said display unit displays an identifier indicating the output state and an identifier indicating the copy state.

9. The system according to claim 7, further comprising a calculation unit configured to calculate a total value of radiation doses at the time of generating a radiation image stored in said storage unit,
wherein said calculation unit calculates the total value of the radiation doses based on image information added to a radiation image which is not determined as a copy image based on a copy state included in the image information, and
said display unit displays a total value calculated by said calculation unit.

10. The system according to claim 7, further comprising a selection unit configured to select one of radiation images displayed by said display unit,
wherein said display unit displays said one radiation image selected by said selection unit.

11. The system according to claim 10, wherein said selection unit selects a radiation image copied by said copying unit.

12. An information processing method for a radiation imaging system for imaging an object to obtain a radiation image based on an imaging protocol, the method comprising:
a storage step of storing a generated radiation image in a storage unit, with image information indicating an attribute of the radiation image being added thereto;
a copying step of, upon receiving an instruction to copy a radiation image stored in the storage unit, copying the radiation image and image information added to the radiation image; and
a changing step of changing an item, of items included in copied image information copied in the copying step, which is unique to each of radiation images different from each other, in accordance with a copied image, thereby changing the copied image information,
wherein in the copying step, methods of copying an imaging protocol used for obtaining a radiation image for which the copy instruction has been issued are switched, in accordance with a relationship between the radiation image and the imaging protocol.

13. A non-transitory computer-readable storage medium storing a computer program for causing a computer to function as each unit of a radiation imaging system defined in claim 1.

14. A control apparatus for X-ray imaging, the apparatus comprising:
an obtaining unit configured to obtain imaging information concerning X-ray imaging;
a reception unit configured to receive an X-ray image captured in accordance with the imaging information from an X-ray imaging unit; and
a display control unit configured to display, on a window, a first image indicating the received X-ray image, imaging information corresponding to the first image, and an icon for reception of an operation input to issue an instruction to copy the X-ray image, and to display, on the window, a second image based on the received X-ray image in response to the reception of the operation input.

15. The apparatus according to claim 14, further comprising a copying unit configured to copy the received X-ray image in accordance with the operation input.

16. The apparatus according to claim 14, wherein said display control unit displays a reduced image of the received X-ray image as the first image and a reduced image of the X-ray image obtained by copying as the second image.

17. The apparatus according to claim 15, wherein said copying unit obtains second imaging information based on first imaging information corresponding to the received X-ray image in accordance with the operation input.

18. The apparatus according to claim 17, wherein said copying unit assigns the first imaging information identification information different from that of the second imaging information.

19. The apparatus according to claim 14, wherein said display control unit displays, on the window, a first image indicating the received X-ray image and first imaging information corresponding to the first image, and also displays, on the window, a second image based on the received X-ray image and second imaging information corresponding to the second image in accordance with the operation input.

20. The apparatus according to claim 14, wherein said display control unit display, on the window, the first image and second image in association with the imaging information.

21. The apparatus according to claim 14, wherein said display control unit displays, in a specific area on the window, imaging information which has not undergone imaging, imaging information which has undergone imaging, the first image indicating an X-ray image corresponding to imaging information, and the second image indicating an X-ray image obtained by copying.

22. The apparatus according to claim 1, wherein the imaging protocol includes an imaging condition for imaging an object and an image processing condition for processing a radiation image.
